(19) United States
(12) Patent Application Publication
(10) Pub. No.: US 2006/0153772 A1
(43) Pub. Date: Jul. 13, 2006

(54) Contextual fear conditioning for predicting immunotherapeutic efficacy

(75) Inventor: Jack Steven Jacobsen, Ramsey, NJ (US)

Correspondence Address: LAHIVE & COCKFIELD, 28 STATE STREET, BOSTON, MA, 02109, US

(73) Assignee: Wyeth, Madison, NJ (US)

(21) Appl. No.: 11/305,889

(22) Filed: Dec. 15, 2005

Related U.S. Application Data
(60) Provisional application No. 60/636,842, filed on Dec. 15, 2004.
(60) Provisional application No. 60/637,253, filed on Dec. 16, 2004.
(60) Provisional application No. 60/736,119, filed on Nov. 10, 2005.

(51) Int. Cl. A61K 49/00

(52) U.S. Cl. 424/9.2

(57) ABSTRACT

The invention provides methods for determining effective immunotherapeutic agents which may be used for the treatment of cognitive disorders.

### RELATED APPLICATIONS

This application is related to co-pending U.S. provisional patent applications bearing Ser. No. 60/736,119 (filed Nov. 10, 2005), Ser. No. 60/636,842 (filed Dec. 15, 2004) and Ser. No. 60/637,253 (filed Dec. 16, 2004), all entitled “Contextual Fear Conditioning for Predicting Immunotherapeutic Efficacy”. The entire content of the above-referenced applications are incorporated herein by reference.

### BACKGROUND OF THE INVENTION

Memory is a key cognitive function involving the storage and/or retrieval by the brain of information received from past experiences. Learning, also referred to as conditioning, is the process by which new information is acquired and stored by the nervous system to form a memory. In patients with dementia, the cognitive pathways for learning and/or memory are impaired, such that the patient fails to learn or effectively form new memories or recall old ones. The number of individuals exhibiting dementia is rising rapidly, and the rate of rise is expected to increase as the general population continues to age and life expectancy continues to lengthen. Patients with dementia require increasingly costly and intensive caregiving as their symptoms worsen. As such, medical interventions that delay institutionalization would help reduce the demands on healthcare systems, in addition to alleviating the sufferings of the subject with the dementia.

The development of profound dementia is characteristic of several amyloidogenic disorders noted for the accumulation of amyloid protein deposits in the brain tissue of affected subjects, including Down's syndrome, cerebral amyloid angiopathy, vascular dementias, and Alzheimer's disease (AD). AD is a progressive disease resulting in senile dementia. Broadly speaking, the disease falls into two categories: late onset, which occurs in old age (65+years) and early onset, which develops well before the senile period, i.e., between 35 and 60 years. Neurodegeneration is associated with amyloidogenic disorders and other dementia disorders such that the cognitive symptoms progressively worsen with age. The diagnosis of an amyloidogenic disorder can usually only be confirmed by the distinctive cellular pathology that is evident on post-mortem examination of the brain. The histopathology consists of at least one of three principal features including the presence of neurofibrillary tangles (NT), the diffuse loss of synapses and neurons in central nervous system tissues, and the presence of amyloid plaques (also called senile plaques). See generally Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995).

The principal constituent of the plaques is a peptide termed Aβ or β-amyloid peptide. Aβ peptide is an approximately 4-kDa internal fragment of 39-43 amino acids of a larger transmembrane glycoprotein named protein termed amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize AD.

Mouse models have been used successfully to determine the significance of amyloid plaques in AD (Games et al., supra, Johnson-Wood et al., Proc. Natl. Acad. Sci. USA 94:1550 (1997)). In particular, when PDAPP transgenic mice, (which express a mutant form of human APP and develop AD pathology at a young age), are injected with the long form of Aβ, they display both a decrease in the progression of AD Pathology and an increase in antibody titers to the Aβpeptide (Schenk et al., Nature 400, 173 (1999)). The above findings implicate Aβ, particularly in its long form, as a causative element in AD.

Aβ peptide can exist in solution and can be detected in the central nervous system (CNS) (e.g., in cerebral spinal fluid (CSF)) and plasma. Under certain conditions, soluble Aβ is transformed into fibrillary, toxic, β-sheet forms found in neuritic plaques and cerebral blood vessels of patients with AD. Several treatments have been developed which attempt to prevent the formation of Aβ peptide, for example, the use of chemical inhibitors to prevent the cleavage of APP. Immunotherapeutic treatments have also been investigated as a means to reduce the density and size of existing plaques. These strategies include passive immunization with various anti-Aβ antibodies that induce clearance of amyloid deposits, as well as active immunization with soluble forms of Aβ peptide to promote a humoral response that includes generation of anti-Aβ antibodies and cellular clearance of the deposits. Both active and passive immunization have been tested as in mouse models of AD. In PDAPP mice, immunization with Aβ was shown to prevent the development of plaque formation, neuritic dystrophy and astrogliosis. Treatment of older animals also markedly reduced the extent and progression of these AD-like neuropathologies (Schenk et al., supra). Aβ immunization was also shown to reduce plaques and behavioral impairment in the TgCRND8 murine model of AD (Janus et al. Nature 408:979-982 (2000)). Aβ immunization also improved cognitive performance and reduced amyloid burden in Tg 2576 APP/PS 1 mutant mice (Morgan et al. Nature 408:982-985 (2000)). Passive immunization of PDAPP transgenic mice has also been investigated. It was found, for example, that peripherally administered antibodies enter the central nervous system (CNS) and induced plaque clearance in vivo (Bard et al. Nat. Med. 6:916-919 (2000)). The antibodies were further shown to induce Fc receptor-mediated phagocytosis in an ex vivo assay. Antibodies specific for the N-terminus of Aβ42 have been demonstrated to be particularly effective in reducing plaque both ex vivo and in vivo (see U.S. Pat. No. 6,761,888 and Bard et al. Proc. Natl. Acad. Sci. USA 100:2023-2028 (2003)). Antibodies specific for the mid-region of Aβ42 also showed efficacy (see e.g., U.S. Pat. No. 6,761,888 and International Patent Application WO/0072880).

Two mechanisms are proposed for effective plaque clearance by immunotherapeutics, i.e., central degradation and peripheral degradation. The central degradation mechanism relies on antibodies being able to cross the blood-brain barrier, bind to plaques, and induce clearance of pre-existing plaques. Clearance has been shown to be promoted through an Fc-receptor-mediated phagocytosis (Bard, et al. Nat. Med. 6:916-19 (2000)). The peripheral degradation mechanism of Aβ clearance relies on a disruption of the dynamic equilibrium of Aβ between brain, CSF, and plasma by anti-Aβ antibodies, leading to transport of Aβ from one compartment to another. Centrally derived Aβ is transported into the CSF and the plasma where it is degraded. Recent studies have concluded that soluble and unbound Aβ are involved in the memory impairment associated with AD, even without reduction in amyloid deposition in the brain. Further studies are needed to determine the action and/or interplay of these pathways for Aβ clearance (Dodel, et al., The Lancet, 2:215 (2003)).

While the majority of treatments to date have been aimed at reducing amyloid plaque buildup, it has been recently noted that certain cognitive impairments (e.g. hippocampal-dependent conditioning defects) associated with amyloidogenic disorders begin to appear before amyloid deposits and gross neuropathology are evident (Dineley et al., J. Biol. Chem., 227: 22768 (2002)). Furthermore, while the pathogenic role of amyloid peptide aggregated into plaques has been known for many years, the severity of dementia or cognitive deficits is only somewhat correlated with the density of plaques whereas a significant correlation exists with the levels of soluble Aβ (see, e.g., McLean et al., Ann Neurol, 46:860-866 (1999)). Some studies have shown or suggested that soluble Aβ oligomers are implicated in synaptotoxicity and memory impairment in APP transgenic mice due to mechanisms such as increased oxidative stress and induction of programmed cell death. (See, e.g., Lambert, et al., PNAS, 95: 6448-53 (1998); Naslund et al., JAMA, 283: 1571 (2000); Mucke et al., J Neurosci, 20:4050-4058 (2000); Morgan et al., Nature, 408:982-985 (2000); Dodart et al., Nat Neurosci, 5:452-457 (2002); Selkoe et al., (2002), Science, 298: 789-91; Walsh et al., Nature, 416:535-539 (2002)). These results indicate that neurodegeneration may begin prior to, and is not solely the result of, amyloid deposition. Therefore, it is desirous to investigate therapeutic strategies which are able to inhibit or reverse the progression of the dementia and/or cognitive deficit associated with an amyloidogenic disease, prior to the significant accumulation of amyloid deposits.

### SUMMARY OF THE INVENTION

The instant invention fulfills a longstanding need for methods of identifying immunotherapeutic agents that are effective in preventing or ameliorating the dementia and/or cognitive deficit that is associated in patients with cognitive disorders such as Alzheimer's disease. A featured aspect of the present invention provides assay methods that are predictive of efficacious therapeutic agents that intervene early in the disease pathogenesis and prevent irreversible neural damage and dementia. The methods of the invention may be utilized to identify immunotherapeutic agents that are effective for improving cognition is a subject suffering from a cognitive disorder. In particular, the methods may be utilized to identify immunotherapeutic agents that are effective for rapid improvement of cognition in a subject.

In one aspect, the invention provides a method for identifying an immunotherapeutic agent effective for improving cognition (e.g., rapidly improving cognition) in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
   whereby an improvement in the context-dependent memory of the animal identifies the immunotherapeutic agent as effective for improving cognition in the subject.

In another aspect, the invention provides a method for identifying an immunotherapeutic agent effective for improving cognition (e.g., rapidly improving cognition) in a subject suffering from a cognitive disorder, comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to an appropriate control,
   whereby an improvement in context-dependent fear response identifies the immunotherapeutic agent as effective for improving cognition in the subject.

In another aspect, the invention provides a method for identifying an immunotherapeutic agent effective for improving cognition (e.g., rapidly improving cognition) in a subject suffering from a cognitive disorder, the method comprising the steps of:
(i) administering an Aβ peptide to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus when the in vivo concentration of the test immunotherapeutic agent is more than 50% of the dose administered in step (i),
   whereby an improvement in context-dependent memory identifies the immunotherapeutic agent as effective in improving cognition in the subject.

In another aspect, the invention provides a method for identifying an immunotherapeutic agent effective for improving cognition (e.g., rapidly improving cognition) in a subject suffering from a cognitive disorder, the method comprising the steps of:
(i) administering an Aβ peptide to a model animal of an amyloidogenic disorder wherein the model animal exhibits a cognitive deficit;
(ii) conducting at least one training session in which the model animal is administered a context-dependent stimulus that is paired with an aversive stimulus; and
(iv) conducting at least one testing session in which the model animal is administered a context-dependent stimulus when the in vivo concentration of the test immunotherapeutic agent is more than 50% of the dose administered in step (i),
   whereby an improvement in context-dependent memory identifies the immunotherapeutic agent as effective in improving cognition in the subject.

In another aspect, the invention provides a method for identifying an immunotherapeutic agent effective for improving cognition (e.g., rapidly improving cognition) in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a wild-type animal administered the test immunotherapeutic,
   whereby a nonsignificant difference in status of impairment of the model animal as compared to the wild-type animal identifies the test immunotherapeutic agent as effective in improving cognition in the subject.

In another aspect, the invention provides a method for identifying an immunotherapeutic agent effective for improving cognition (e.g., rapidly improving cognition) in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a model animal that is not administered the test immunotherapeutic agent,
   whereby a significant difference in deficit reversal of the model animal in step (iii) as compared to the model animal that is not administered the test immunotherapeutic agent identifies the test immunotherapeutic agent as effective in improving cognition.

In another aspect, the invention provides a method for identifying an immunotherapeutic agent effective for improving cognition (e.g., rapidly improving cognition) in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a wild-type animal administered the test immunotherapeutic;
(v) comparing a context-dependent fear response of the model animal in step (iii) to a context-dependent fear response of a model animal that is not administered the test immunotherapeutic agent,
   whereby a nonsignificant difference in status of impairment of the model animal as compared to the wild-type animal and a significant difference in deficit reversal of the model animal in step (iii) as compared to the model animal that is not administered the test immunotherapeutic agent identifies the test immunotherapeutic agent as effective in improving cognition.

The methods of the invention can be used to identify an immunotherapeutic that is effective in treating any subject suffering from a cognitive disorder. In one embodiment the cognitive disorder is a dementia disorder. In another embodiment, the cognitive disorder is a neurodegenerative disease. In another embodiment, the cognitive disorder is an amyloidogenic disorder. In exemplary embodiments, the cognitive disorder is an Aβ-related cognitive disorder. In another exemplary embodiment, the cognitive disorder is an Aβ-related dementia disorder. In another exemplary embodiment, the cognitive disorder is Alzheimer's disease.

Any model animal which exhibits symptoms of an amyloidogenic disorder or is genetically predisposed to develop symptoms of an amyloidogenic disorder is a suitable model animal for use in the methods of the invention. In preferred embodiments, the model animal exhibits a cognitive impairment. In certain embodiments, the cognitive deficit is impairment in procedural learning and/or memory. In other embodiments, the impairment in procedural learning and/or memory is contextual-dependent. In another embodiment, the impairment in procedural learning and/or memory is cue-dependent. In exemplary embodiments, the model animal is a transgenic mouse containing a mutation in an Alzheimer's related gene. In another exemplary embodiment, the model animal is selected from the group consisting of a PDAPP mouse, a Tg2576 mouse, a TgAPP22 mouse, a TgAPP/LD/2 mouse, a PSEN-1 A246E mouse, a PSEN-1 DeltaE9 mouse, a Tg2576+PSEN-1 mouse, a TgHu/MoAPP A246E +PSEN-1 mouse, a TgHu/MoAPP DeltaE9 +PSEN-1 mouse, a TgCDNR8 mouse, a PSAPP mouse, and a 3×Tg-AD mouse.

The methods of the invention are not limited to the use of an animal of a particular age, although in certain embodiments, the model animal is at least 20 weeks of age prior to administration of the test immunotherapeutic agent. In other embodiments, the model animal is at least 10 weeks of age, prior to administration of the test immunotherapeutic agent

The methods of the invention involve a training session during which a model animal that is administered a test immunotherapeutic agent is conditioned to an aversive stimulus. In preferred embodiments, two or fewer training sessions are suitable to condition the model animal to the aversive stimulus. In certain embodiments the aversive stimulus administered during the training session is a footshock. In other embodiments, the context-dependent stimulus administered during the training session is an altered cage. In certain embodiments, a cue-dependent stimulus is also administered to the model animal during the training phase. In exemplary embodiments, the cue-dependent stimulus is an auditory stimulus. In another embodiment, the aversive stimulus is paired with the context-dependent stimulus or the cue-dependent stimulus.

The methods of the invention also involve a testing session during which the effects of a test immunotherapeutic agent on the cognitive function of the model animal are evaluated by administering a context-dependent stimulus in the absence of the aversive stimulus and measuring the fear response of the animal. In preferred embodiments, the training session is administered to the model animal within 24 hours following the administration of the immunotherapeutic agent. In certain embodiments, the context-dependent stimulus administered during the training session is an altered cage. In other embodiments, a cue-dependent stimulus is also administered to the model animal during the testing phase in the absence of the aversive stimulus. In exemplary embodiments, the cue-dependent stimulus is an auditory stimulus. In another exemplary embodiment, the fear response is a freezing behavior.

Improvements in the cognition of a model animal may be evaluated by comparing the fear response of the model animal to a suitable control. In one embodiment, the suitable control is a wild-type animal administered the test immunotherapeutic. In another embodiment, suitable control is a model animal that is not administered the test immunotherapeutic agent. In another embodiment, an improvement in cognition of a model animal is indicated by a nonsignificant difference in the fear response of a model animal as compared to a wild-type animal that is administered the immunotherapeutic agent. In another embodiment, an improvement in cognition of a model animal is indicated by a significant difference in fear response of the model animal as compared to a model animal that is not administered the test immunotherapeutic agent.

The steps of the methods of the invention may be repeated systematically with different test immunotherapeutic agents. Alternatively, steps may be repeated with the same immunotherapeutic agent in an iterative process whereby the selection of the test immunotherapeutic agent as an effective immunotherapeutic agent is validated or the potency of the test immunotherapeutic agent is determined by repeating steps with a higher or lower concentration of test immunotherapeutic agent. In one exemplary embodiment, steps are repeated one to five times with increasingly lower concentrations of immunotherapeutic agent.

In certain embodiments, the animal model is administered multiple doses of a test immunotherapeutic agent.

The timing or interval of time between steps is not considered to be limiting. However, in a preferred embodiment, the testing step is performed within 24 hours of the administration step.

The test immunotherapeutic agent may be any active immunotherapeutic agent (e.g. an antigen or immunogen) or passive immunotherapeutic agent (e.g. antibody). Cells expressing the active or passive immunotherapeutic are also within the scope of the invention. In one exemplary embodiment, the test immunotherapeutic agent is an immunogenic preparation of an Aβ peptide. In another preferred embodiment, the test immunotherapeutic agent is an Aβ antibody.

In an additional aspect, the invention provides a method for identifying an immunotherapeutic agent effective in neutralizing one or more toxic soluble forms of Aβ peptide, comprising the steps of:
(i) administering a test immunotherapeutic agent to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
   whereby an improvement in context-dependent memory identifies the immunotherapeutic agent as effective in neutralizing one or more toxic soluble forms of Aβ peptide.

In one embodiment, the test immunotherapeutic agent is subsequently tested for its ability to bind to and/or clear insoluble forms of Aβ peptide. In another embodiment, a greater than 50% reduction in the size and number of amyloid deposits identifies the immunotherapeutic agent as effective in clearing plaque.

In another aspect, the invention provides a method of identifying an epitope in an Aβ peptide, the method comprising the steps of:
(i) administering an Aβ peptide to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
   whereby an improvement in context-dependent memory identifies an epitope in the Aβ peptide.

In one embodiment, the method is repeated with a truncated form of the Aβ peptide. In another embodiment, the Aβ peptide is used as an active immunotherapeutic agent for improving cognition in a subject. In another embodiment, the Aβ peptide used to generate an antibody. In another embodiment, the antibody is used as a passive immunotherapeutic agent for improving cognition in a subject.

### BRIEF DESCRIPTION OF THE DRAWINGS

FIGS. 1A-B depict the effect of an anti-Aβ antibody, 12A11, on contextual-dependent memory in wild-type and Tg2576 mice as determined by a contextual fear conditioning (CFC) assay.

FIG. 2 depicts the effect of the N-terminal anti-Aβ antibodies (3D6, 12A11, and 266), on contextual-dependent memory in wild-type and Tg2576 mice as determined by a contextual fear conditioning (CFC) assay.

FIG. 3 depicts the effect of the central anti-Aβ antibody 266, on contextual-dependent memory in wild-type and Tg2576 mice as determined by a contextual fear conditioning (CFC) assay.

FIG. 4 depicts the effect of anti-Aβ antibodies (12A11 and 266), on contextual-dependent memory in wild-type and 18-20 month old, double transgenic AD mice as determined by a contextual fear conditioning (CFC) assay.

### DETAILED DESCRIPTION OF THE INVENTION

The invention is based, at least in part, on the development of a model animal-based assay methodology that is highly predictive of immunotherapeutic efficacy in humans. The methodology of the invention is particularly effective in determining the ability of the immunotherapeutic agents to reverse deficits in both explicit and implicit long-term memory.

### 1) DEFINITIONS

Prior to describing the invention, it may be helpful to have an understanding thereof to set forth definitions of certain terms to be used hereinafter.

The term “Aβ-related disease or disorder” as used herein refers to a disease or disorder associated with, or characterized by, the development or presence of an Aβ peptide. In one embodiment, the Aβ-related disease or disorder is associated with or characterized by the presence of soluble Aβ. In another embodiment, the Aβ-related disease or disorder is associated with or characterized by the presence of insoluble Aβ. In another embodiment, the Aβ-related disease or disorder is associated with or characterized by the presence of a neuroactive Aβ species (NAβ). In another embodiment, the Aβ-related disease or disorder is also an amyloidogenic disorder. In another embodiment, the Aβ-related disease or disorder is characterized by an Aβ-related cognitive deficit or disorder, for example, an Aβ-related dementia disorder. Exemplary Aβ-related diseases or disorders include Alzheimer's disease (AD), Down's syndrome, cerebral amyloid angiopathy, certain vascular dementias, and mild cognitive impairment (MCI).

The terms “β-amyloid protein”, “β-amyloid peptide”, “β-amyloid”, “Aβ” and “Aβ peptide” are used interchangeably herein. Aβ peptide (e.g., Aβ39, Aβ40, Aβ41, Aβ42 and Aβ43) is a ˜4-kDa internal fragment of 39-43 amino acids of the larger transmembrane glycoprotein termed Amyloid Precursor Protein (APP). Multiple isoforms of APP exist, for example APP⁶⁹⁵, APP⁷⁵¹, and APP⁷⁷⁰. Amino acids within APP are assigned numbers according to the sequence of the APP⁷⁷⁰ isoform (see e.g., GenBank Accession No. P05067). Examples of specific isotypes of APP which are currently known to exist in humans are the 695 amino acid polypeptide described by Kang et. al. (1987) Nature 325:733-736 which is designated as the “normal” APP; the 751 amino acid polypeptide described by Ponte et al. (1988) Nature 331:525-527 (1988) and Tanzi et al. (1988) Nature 331:528-530; and the 770-amino acid polypeptide described by Kitaguchi et. al. (1988) Nature 331:530-532. As a result of proteolytic processing of APP by different secretase enzymes in vivo or in situ, Aβ is found in both a “short form”, 40 amino acids in length, and a “long form”, ranging from 42-43 amino acids in length. The short form, Aβ₄₀, consists of residues 672-711 of APP. The long form, e.g., Aβ₄₂ or Aβ₄₃, consists of residues 672-713 or 672-714, respectively. Part of the hydrophobic domain of APP is found at the carboxy end of Aβ, and may account for the ability of Aβ to aggregate, particularly in the case of the long form. Aβ peptide can be found in, or purified from, the body fluids of humans and other mammals, e.g. cerebrospinal fluid, including both normal individuals and individuals suffering from amyloidogenic disorders.

The terms “β-amyloid protein”, “β-amyloid peptide”, “β-amyloid”, “Aβ” and “Aβ peptide” include peptides resulting from secretase cleavage of APP and synthetic peptides having the same or essentially the same sequence as the cleavage products. Aβ peptides can be derived from a variety of sources, for example, tissues, cell lines, or body fluids (e.g. sera or cerebrospinal fluid). For example, an Aβ can be derived from APP-expressing cells such as Chinese hamster ovary (CHO) cells stably transfected with APP_{717V→F}, as described, for example, in Walsh et al., (2002), Nature, 416, pp 535-539. An Aβ preparation can be derived from tissue sources using methods previously described (see, e.g., Johnson-Wood et al., (1997), Proc. Natl. Acad. Sci. USA 94:1550). Alternatively, Aβ peptides can be synthesized using methods which are well known to those in the art. See, for example, Fields et al., Synthetic Peptides: A User's Guide, ed. Grant, W.H. Freeman & Co., New York, N.Y., 1992, p 77). Hence, peptides can be synthesized using the automated Merrifield techniques of solid phase synthesis with the α-amino group protected by either t-Boc or F-moc chemistry using side chain protected amino acids on, for example, an Applied Biosystems Peptide Synthesizer Model 430A or 431. Longer peptide antigens can be synthesized using well known recombinant DNA techniques. For example, a polynucleotide encoding the peptide or fusion peptide can be synthesized or molecularly cloned and inserted in a suitable expression vector for the transfection and heterologous expression by a suitable host cell. Aβ peptide also refers to related Aβ sequences that results from mutations in the Aβ region of the normal gene.

The term “soluble Aβ” or “dissociated Aβ” refers to non-aggregating or disaggregated Aβ polypeptide, including monomeric soluble as well as oligomeric soluble Aβ polypeptide (e.g., soluble Aβ dimers, trimers, and the like). Soluble Aβ can be found in vivo in biological fluids such as cerebrospinal fluid and/or serum. Soluble Aβ can also be prepared in vitro, e.g., by solubilizing Aβ peptide in appropriate solvents and/or solutions. For example, soluble Aβ can be prepared by dissolving lyophilized peptide in alcohol, e.g., HFIP followed by dilution into cold aqueous solution. Alternatively, soluble Aβ can be prepared by dissolving lyophilized peptide in neat DMSO with sonication. The resulting solution can be centrifuged (e.g., at 14,000×g, 4° C., 10 minutes) to remove any insoluble particulates.

The term “insoluble Aβ” or “aggregated Aβ” refers to aggregated Aβ polypeptide, for example, Aβ held together by noncovalent bonds and which can occur in the fibrillary, toxic, β-sheet form of Aβ peptide that is found in neuritic plaques and cerebral blood vessels of patients with AD. Aβ(e.g., Aβ42) is believed to aggregate, at least in part, due to the presence of hydrophobic residues at the C-terminus of the peptide (part of the transmembrane domain of APP).

As used herein, the phrase “neuroactive Aβ species” refers to an Aβ species (e.g., an Aβ peptide or form of Aβ peptide) that effects at least one activity or physical characteristic of a neuronal cell. Neuroactive Aβ species effect, for example, the function, biological activity, viability, morphology and/or architecture of a neuronal cell. The effect on neuronal cells can be cellular, for example, effecting the long-term-potentiation (LPT) of a neuronal cell or viability of a neuronal cell (neurotoxicity). Alternatively, the effect can be on an in vivo neuronal system, for example, effecting a behavioral outcome in an appropriate animal test (e.g., a cognitive test). The term “neutralize” as used herein means to make neutral, counteract or make ineffective an activity or effect.

As used herein, the term “neurodegenerative disease” refers broadly to disorders or diseases associated with or characterized by degeneration of neurons and/or nervous tissues, e.g. an amyloidogenic disease.

The term “amyloidogenic disease” or “amyloidogenic disorder” includes any disease associated with (or caused by) the formation or deposition of insoluble amyloid fibrils. Exemplary amyloidogenic diseases include, but are not limited to systemic amyloidosis, Alzheimer's disease (AD), cerebral amyloid angiopathy (CAA), mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the principal polypeptide component of the amyloid deposit. Accordingly, Alzheimer's disease is an example of a “disease characterized by deposits of Aβ” or a “disease associated with deposits of Aβ”, e.g., in the brain of a subject or patient. Other diseases characterized by deposits of Aβ can include uncharacterized diseases where amyloidogenic deposits are found in one or more regions of the brain associated with learning and/or memory, e.g., the hippocampus, amygdala, subiculum, cingulated cortex, prefrontal cortex, perirhinal cortex, sensory cortex, and medial temporal lobe.

The term “cognition” refers to cognitive mental processes performed by a subject including, but not limited to, learning or memory (e.g., short-term or long term learning or memory), knowledge, awareness, attention and concentration, judgement, visual recognition, abstract thinking, executive functions, language, visual-spatial (i.e., visuo-spatial orientation) skills, visual recognition, balance/agility and sensorimotor activity. Exemplary cognitive processes include learning and memory.

The terms “cognitive disorder”, “cognitive deficit”, or “cognitive impairment” are used interchangeably herein and refer to a deficiency or impairment in one or more cognitive mental processes of a subject. Cognitive deficits may have a number of origins: a functional mechanism (anxiety, depression), physiological aging (age-associated memory impairment), brain injury, psychiatric disorders (e.g. schizophrenia), drugs, infections, toxicants, or anatomical lesions. Exemplary cognitive deficits include deficiency or impairment in learning or memory (e.g., in short-term or long term learning and/or memory loss of intellectual abilities, judgment, language, motor skills, and/or abstract thinking).

As used herein, the term “Aβ-related cognitive disorder” (or “deficit” or “impairment”) refers to a cognitive disorder associated with, or characterized by, the development or presence of an Aβ peptide. In one embodiment, the Aβ-related disease or disorder is associated with or characterized by the presence of soluble Aβ. In another embodiment, the Aβ-related disease or disorder is associated with or characterized by the presence of insoluble Aβ. In another embodiment, the Aβ-related disease or disorder is associated with or characterized by the presence of a neuroactive Aβ species (NAβ).

The term “dementia disorder”, as used herein, refers to a cognitive disorder characterized by dementia (i.e., general deterioration or progressive decline of cognitive abilities or dementia-like symptoms). Dementia disorders are often associated with, or caused by, one or more aberrant processes in the brain or central nervous system (e.g. neurodegeneration). Dementia disorders commonly progress from mild through severe stages and interfere with the ability of a subject to function independently in everyday life. Dementia may be classified as cortical or subcortical depending on the area of the brain affected. Dementia disorders do not include disorders characterized by a loss of consciousness (as in delirium) or depression, or other functional mental disorders (pseudodementia). Dementia disorders include the irreversible dementias such as those associated with neurodegenerative diseases such Alzheimer's disease, vascular dementia, Lewy body dementia, Jakob-Creutzfeldt disease, Pick's disease, progressive supranuclear palsy, Frontal lobe dementia, idiopathic basal ganglia calcification, Huntington disease, multiple sclerosis, and Parkinson's disease, as well as reversible dementias due to trauma (posttraumatic encephalopathy), intracranial tumors (primary or metastatic), subdural hematomas, metabolic and endocrinologic conditions (hypo- and hyperthyroidism, Wilson's disease, uremic encephalopathy, dialysis dementia, anoxic and post-anoxic dementia, and chronic electrolyte disturbances), deficiency states (Vitamin B12 deficiency and pellagra (vitamin B6)), infections (AIDS, syphilitic meningoencephalitis, limbic encephalitis, progressive multifocal leukoencephalopathy, fungal infections, tuberculosis), and chronic exposure to alcohol, aluminum, heavy metals (arsenic, lead, mercury, manganese), or prescription drugs (anticholinergics, sedatives, barbiturates, etc.).

As used herein, the term “Aβ-related dementia disorder” refers to a dementia disorder associated with, or characterized by, the development or presence of an Aβ peptide.

As used herein, the phrase “improvement in cognition” refers to an enhancement or increase in a cognitive skill or function. Likewise, the phrase “improving cognition” refers to the enhancing or increasing of a cognitive skill or function. An improvement in cognition is relative, for example, to cognition in the subject before a treatment according to the instant invention. Preferably, the improvement in cognition trends towards that of a normal subject or towards a standard or expected level.

The term “rapid”, as used, for example, in the phrase “rapid improvement in cognition” (or “rapidly improving cognition”) means taking a relatively or comparatively short time or occurring within a comparatively short time interval; i.e., that an effect (e.g., improvement) is accomplished, observed or achieved comparatively quickly with respect to a suitable control.

An exemplary “rapid improvement in cognition” is accomplished, observed or achieved within one day (i.e., within 24 hours). A “rapid improvement in cognition” may be accomplished, observed or achieved in less than one day (i.e., less than 24 hours), for example, within 23, 22, 21, 20, 29, 18, 17, 16, 15, 14, 13, 12, 11, 10, 9, 8, 7, 6, 5, 4, 3, 2 or 1 hour(s). A “rapid improvement in cognition” may alternatively be accomplished, observed or achieved in more than one day but preferably within one month, for example, within 31, 30, 29, 28, 27, 26, 25, 24, 23, 22, 21, 20, 19, 18, 17, 16, 15, 14, 13, 12, 11, 10,9, 8, 7,6, 5,4, 3 or 2 days. Exemplary time intervals for accomplishing, observing or achieving a rapid improvement in cognition are within weeks, e.g., within three weeks, within two weeks or within one week or within, for example, 120 hours, 96 hours, 72 hours, 48 hours, 24 hours, 18 hours, 12 hours and/or 6 hours.

The term “prolonged”, as used, for example, in the phrase “prolonged improvement in cognition” means occurring over a comparatively or relatively longer time interval than a suitable control; i.e., that a desired effect (e.g., improvement) occurs or is observed to be sustained without interruption for an extended or protracted time period with respect to a suitable control.

An exemplary “prolonged improvement in cognition” is accomplished, observed or achieved for at least one week. A “prolonged improvement in cognition” may be accomplished, observed or achieved for more than one day (i.e., more than 24 hours), for example, for more than 36 hours, 48 hours (i.e., 2 days), 72 hours (i.e., 3 days), 96 hours (i.e., 4 days) 108 hours (i.e., 5 days) or 132 hours (i.e., 6 days). A “prolonged improvement in cognition” may alternatively be accomplished, observed or achieved for more than one week, e.g., for 8, 9, 10, 11, 12, 13, or 14 days (i.e., two weeks), three weeks, four weeks, five weeks, six weeks, or more. Exemplary time intervals over which a prolonged improvement in cognition is accomplished, observed or achieved include 5, 6, 7, 8, 9, 10, 11, 12, 13, 14, 15, 16, 17, 18, 19 or 20 days.

The term “modulation” as used herein refers to both upregulation, i.e. stimulation, and downregulation, i.e. suppression, of a response.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use upon the severity of the disease, the patient's general physiology. e.g., the patient's body mass, age, gender, the route of administration, and other factors well known to physicians and/or pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). An effective dose of antibody used in the present methods will range, for example, between 1 μg/kg and 500 mg/kg. An exemplary range for effective doses of antibodies used in the methods of the present invention is between 0.1 mg/kg and 100 mg/kg. Exemplary effective doses include, but are not limited to, 10 μg/kg, 30 μg/kg, 100 μg/kg, 300 μg/kg, 1 mg/kg, 30 mg/kg and 100 mg/kg.

As used herein, the term “administering” refers to the act of introducing a pharmaceutical agent into a subject's body. An exemplary route of administration in the parenteral route, e.g., subcutaneous, intravenous or intraperitoneal administration.

The terms “patient” or “subject” are used interchangeably herein and include human and other mammalian subjects that receive either prophylactic or therapeutic treatment with immunotherapeutic agents (e.g. immunotherapeutic agents identified by the methods of the invention).

The term “model animal” or “animal model” as used herein includes a member of a mammalian species such as rodents, non-human primates, sheep, dogs, and cows that exhibit features or characteristics of a certain system of disease or disorder, e.g., a human system, disease or disorder. Exemplary non-human animals selected from the rodent family include rabbits, guinea pigs, rats and mice, most preferably mice. An “animal model” of, or “model animal” having, a dementia disorder exhibits, for example, prominent cognitive deficits associated with a dementia-related disorder (e.g., AD). Preferably the model animal exhibits a progressive worsening of the cognitive deficit with increasing age, such that the disease progression in the model animal parallels the disease progression in a subject suffering from the dementia disorder. In exemplary animal models, the cognitive deficit exhibited by the model animal is deficiency in learning or memory, e.g., learning/memory of spatial orientation, avoidance, or fear. Cognitive deficits can be identified by impaired performance of any of one or more cognitively-based tasks that are well established in the art. Suitable behavioral tests include sensorimotor tasks for example, balance beam tasks, string suspension tasks, and open-field tasks. Additional suitability primarily designed to test learning or memory, for example, Morris water maze tasks (with visible or hidden platforms), radial water maze tasks, elevated plus-maze tasks, circular platform tasks, Y-maze tasks, object recognition tasks, or avoidance tasks (passive or active).

The term “aversive stimulus” is referred to herein as an unconditioned stimulus that would normally be avoided by an animal. Aversive stimuli include noxious odors, loud noises, or uncomfortable textures. An exemplary aversive stimulus for use in the methods of the invention is an electrical shock (e.g., an electrical foot shock).

The term “fear response” is a behavior parameter exhibited by a model animal that is elicited, reduced, or enhanced by an aversive stimulus or a cue-dependent or context-dependent stimulus that has been previously administered with the aversive stimulus. Fear responses may include reduced or enhanced frequency of “freezing” behaviors (absence of movement except for respiration, which may also include eye blink, or change in the nictitating membrane reflex, depending on the test animal selected), crouching, walking, fur licking, face cleaning, stretching, leaning, or tail shaking.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology of the invention, as described herein. In another embodiment, a “suitable control” or “appropriate control” is a value, level, feature, characteristic, property, etc. determined in a subject, e.g., a control or normal subject exhibiting, for example, normal traits. In yet another embodiment, a “suitable control” or “appropriate control” is a predefined value, level, feature, characteristic, property, etc.

In exemplary embodiments, a suitable control refers to an experimental sample or the results or data derived from or associated with an experimental example that establish a suitable baseline or basal level of cognitive performance of a model animal. Preferably, experiments employing the suitable control have been performed under the same or similar experimental conditions as experiments that employ the test article, thereby allowing the experimenter to make a valid comparison of the effect of the test article. Suitable controls for use in the invention include control animals (e.g. wild-type animals or any animal that does not normally exhibit the symptoms or pathology of a dementia disorder) and control treatments (e.g. innocuous compositions or compositions lacking the test immunotherapeutic agent).

As used herein, the term “immunotherapy” refers to a treatment, for example, a therapeutic or prophylactic treatment, of a disease or disorder intended to and/or producing an immune response (e.g., an active or passive immune response).

The term “immunotherapeutic agent” refers to an agent that comprises or consists of one or more immunogens, immunoglobulins, antibodies, antibody fragments, or antibody chains, as defined herein, or combinations thereof, for use in immunotherapy. Accordingly, as used herein, the term “immunotherapeutic agent” also includes nucleic acids encoding immunogens, immunoglobulins, antibodies, antibody fragments, or antibody chains. Such nucleic acids can be DNA or RNA. A nucleic acid segment encoding an immunogen is typically linked to regulatory elements, such as a promoter and enhancer, that allow expression of the DNA segment in the intended target cells of a subject or patient.

The term “test” or “candidate” immunotherapeutic agent refers to an agent being examined or evaluated for efficacy as an immunotherapeutic agent according the assay methods of the invention. In exemplary embodiments, the immunotherapeutic efficacy of an agent is unknown or uncertain.

An “immunogenic agent” or “immunogen” is capable of inducing an immunological response against itself on administration to a patient, optionally in conjunction with an adjuvant. An “immunogenic composition” is one that comprises an immunogenic agent.

The terms “immunogen”, “immunogenic agent”, or “immunogenic preparation” refer to an agent that is capable of inducing an immunological response against itself on administration to a mammal, optionally in conjunction with an adjuvant.

The term “adjuvant” refers to a compound that when administered in conjunction with an immunogen augments the immune response to the immunogen, but when administered alone does not generate an immune response. Adjuvants can augment an immune response by several mechanisms including lymphocyte recruitment, stimulation of B and/or T cells, and stimulation of macrophages.

The term “immunoglobulin” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. It is intended that the term “antibody” encompass any Ig class or any Ig subclass (e.g. the IgG1, IgG2, IgG3, and IgG4 subclasses of IgG) obtained from any source (e.g., in exemplary embodiments, humans and non-human primates, and in additional embodiments, rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG₁, IgG₂, IgG₃, and IgG₄) that have been identified in humans and higher mammals.

The term “IgG subclass” refers to the four subclasses of immunoglobulin class IgG—IgG₁, IgG₂, IgG₃, and IgG₄ that have been identified in humans and higher mammals by the γ heavy chains of the immunoglobulins, γ₁-γ₄, respectively.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen.

The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

As used herein, the term “antigen binding site” refers to a site that specifically binds (immunoreacts with) an antigen (e.g., a cell surface or soluble antigen). Antibodies preferably comprise at least two antigen binding sites. An antigen binding site commonly includes immunoglobulin heavy chain and light chain CDRs and the binding site formed by these CDRs determines the specificity of the antibody. An “antigen binding region” or “antigen binding domain” is a region or domain (e.g., an antibody region or domain that includes an antibody binding site as defines supra.

Immunoglobulins or antibodies can exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding). Binding fragments are produced by recombinant DNA techniques, or by enzymatic or chemical cleavage of intact immunoglobulins. Binding fragments include Fab, Fab′, F(ab′)₂, Fabc, Fv, single chains, and single chain antibodies. Other than “bispecific” or “bifunctional” immunoglobulins or antibodies, an immunoglobulin or antibody is understood to have each of its antigen-binding sites identical. A “bispecific” or “bifunctional antibody” is an artificial hybrid antibody having two different heavy/light chain pairs and two different antigen-binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes).

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “Fc immunoglobulin variant” or “Fc antibody variant” includes immunoglobulins or antibodies (e.g., humanized immunoglobulins, chimeric immunoglobulins, single chain antibodies, antibody fragments, etc.) an altered Fc region. Fc regions can be altered, for example, such that the immunoglobulin has an altered effector function.

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life.

The term “effector molecule” refers to a molecule that is capable of binding to the Fc region of an antibody (e.g., an IgG antibody) including, but not limited to, a complement protein or a Fc receptor.

The term “effector cell” refers to a cell capable of binding to the Fc portion of an antibody (e.g., an IgG antibody) typically via an Fc receptor expressed on the surface of the effector cell including, but not limited to, lymphocytes, e.g., antigen presenting cells and T cells.

The term “Fc region” refers to a C-terminal region of an IgG antibody, in particular, the C-terminal region of the heavy chain(s) of said IgG antibody. Although the boundaries of the Fc region of an IgG heavy chain can vary slightly, a Fc region is typically defined as spanning from about amino acid residue Cys226 to the carboxyl-terminus of a human IgG heavy chain(s).

An “antigen” is an entity (e.g., a proteinaceous entity or peptide) to which an immunoglobulin or antibody (or antigen-binding fragment thereof) specifically binds.

The term “epitope” or “antigenic determinant” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibodies that recognize the same epitope can be identified in a simple immunoassay showing the ability of one antibody to block the binding of another antibody to a target antigen, i.e., a competitive binding assay. Competitive binding is determined in an assay in which the immunoglobulin under test inhibits specific binding of a reference antibody to a common antigen, such as AP. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Exemplary epitopes or antigenic determinants to which an antibody binds can be found within the human amyloid precursor protein (APP), but are preferably found within the Aβ peptide of APP. Exemplary epitopes or antigenic determinants within Aβ, as described herein, are located within the N-terminus, central region, or C-terminus of Aβ.

An “N-terminal epitope”, is an epitope or antigenic determinant comprising residues located within the N-terminus of Aβ peptide. Exemplary N-terminal epitopes include residues within amino acids 1-10 or 1-12 of Aβ, preferably from residues 1-3, 1-4, 1-5, 1-6, 1-7, 2-6, 3-6, or 3-7 of Aβ42. Other exemplary N-terminal epitopes start at residues 1-3 and end at residues 7-11 of Aβ. Additional exemplary N-terminal epitopes include residues 2-4, 5, 6, 7 or 8 of Aβ, residues 3-5, 6, 7, 8 or 9 of Aβ, or residues 4-7, 8, 9 or 10 of Aβ42.

“Central epitopes” are epitopes or antigenic determinants comprising residues located within the central or mid-portion of the Aβ peptide. Exemplary central epitopes include residues within amino acids 13-28, preferably 16-21, 16-22, 16-23, 16-24, 18-21, 19-21, 19-22, 19-23, or 19-24 of Aβ.

“C-terminal epitopes” are epitopes or antigenic determinants comprising residues located within the C-terminus of the Aβ peptide (e.g., within about amino acids 30-40 or 30-42 of Aβ. Additional exemplary epitopes or antigenic determinants include residues 33-40 or 33-42 of Aβ. Such epitopes can be referred to as “C-terminal epitopes”.

When an antibody is said to bind to an epitope within specified residues, such as Aβ 3-7, what is meant is that the antibody specifically binds to a polypeptide containing the specified residues (i.e., Aβ 3-7 in this an example). Such an antibody does not necessarily contact every residue within Aβ 3-7. Nor does every single amino acid substitution or deletion within Aβ 3-7 necessarily significantly affect binding affinity.

The terms “Aβ antibody” and “anti-Aβ” are used interchangeably herein to refer to an antibody that binds to one or more epitopes or antigenic determinants within Aβ protein. Exemplary Aβ antibodies include N-terminal Aβ antibodies, central Aβ antibodies, and C-terminal Aβ antibodies. As used herein, the term “N-terminal Aβ antibody” shall refer to an Aβ antibody that recognizes at least one N-terminal epitope or antigenic determinant. As used herein, the term “central Aβ antibody” shall refer to an Aβ antibody that recognizes at least one central epitope or antigenic determinant. As used herein, the term “C-terminal Aβ antibody” shall refer to an Aβ antibody that recognizes at least one C-terminal epitope or antigenic determinant.

The term “immunological” or “immune” response is the development of a humoral (antibody mediated) and/or a cellular (mediated by antigen-specific T cells or their secretion products) response directed against an antigen in a subject. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, natural killer (“NK”) cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, REF; Tigges, REF). The relative contributions of humoral and cellular responses to the protective or therapeutic effect of an immunogen can be distinguished by separately isolating antibodies and T-cells from an immunized animal or individual and measuring protective or therapeutic effect in a second subject.

### II) CONTEXTUAL FEAR CONDITIONING (CFC) ASSAY

Several cognitively-based behavioral tests or assays have been developed to assess cognitive performance of model animals which present with symptoms or pathology of dementia disorders. Cognitive deficits can be identified by impaired performance of these tests, and many treatments have been proposed based on their ability to improve performance in these tests. Although some tasks have evaluated behaviors or motor function of these model animals, most tasks have been designed to test learning or memory of the model animal.

The present invention provides a novel method for identifying effective therapeutics for treatment of a dementia disorder using a contextual fear conditioning (CFC) behavioral test paradigm. The method tests the ability of a model animal to acquire fear in response to an aversive conditional stimulus in a context or location where the animal experienced the aversive stimulus (“contextual memory”). The assay can also be used to test the ability of a model animal to acquire fear in response to a non-averse conditional stimulus paired with an aversive stimulus (“cue-dependent memory”). The methods of invention are well-suited for evaluating test immunotherapeutic agents in animal models of dementia disorders and selecting immunotherapeutic agents effective in treating patients suffering from related cognitive diseases. The inventors have discovered that the ability of an immunotherapeutic agent to reverse or prevent a contextual memory deficit of an animal model in a CFC test is predictive of the therapeutic efficacy of that agent in treating an amyloidogenic disorder, e.g. Alzheimer's disease, in a patient.

In the present invention, an exemplary CFC assay is performed according to the following steps 1-7:
Step 1. Placing a test animal in the contextual environment for 2 minutes to allow for acclimatization.
Step 2. Optionally administering a cue to the test animal for 13 seconds.
Step 3. Administering a shock for 2 seconds (optionally paired with a cue).
Step 4. Repeating Steps 1-3.
Step 5. Retaining the test animal in the contextual environment for 30 seconds.
Step 6. Removing the test animal from the contextual environment for 1 day.
Step 7. Returning the test animal to the contextual environment of Step 1 and scoring freezing every 10 seconds for 5 minutes.

In other exemplary embodiments, the CFC assay is performed with the following additional steps 8-10:
Step 8. Removing the test animal from the contextual environment of Step 1 for 1 hour.
Step 9. Placing the test animal an altered contextual environment and scoring freezing behavior for 3 minutes.
Step 10. Administering the cue of step 2 to the animal and scoring freezing behavior for 3 minutes.

The methods of the invention provide several advantages over existing methods. One advantage is that the methods of the invention specifically test the ability of a test immunotherapeutic agent to improve deficits in procedural learning and/or memory, cognitive deficits that are among the most debilitating symptoms of dementia disorders. Unlike explicit memory (also known as “declarative” or “working” memory), which is defined as the ability to store and retrieve specific information that is available to consciousness and which can therefore be expressed by language (e.g. the ability to remember a specific fact or event), procedural memory (also known as “implicit” or “contextual” memory) is the ability to acquire, retain, and retrieve general information or knowledge that is not available to consciousness and which requires the learning of skills, associations, habits, or complex reflexes to be expressed, e.g. the ability to remember how to execute a specific task. Because individuals suffering from dementia with procedural memory deficits are much more impaired in their ability to function normally, the ability to identify a treatment which is effective in improving deficits in procedural memory is highly desirable and advantageous.

The instant invention provides a second advantage in that more than one aspect of procedural memory can be assessed in a single test. The CFC assay paradigm, as described herein, assesses the improvement in both the contextual-dependent and cue-dependent aspects of procedural memory following administration of a test immunotherapeutic agent. This provides the experimenter with the ability to assess the improved function more of than region of the brain, since the context and cue-dependent aspects of procedural require distinct neural processing pathways in the amygdala and hippocampus (Phillips & LeDoux, Behavioral Neurosci., 1992). Indeed, damage to the hippocampus and/or the amygdala (e.g. due to amyloid deposition) has been implicated in amyloidogenic disorders such as Alzheimer's. Since, the CFC assay provides information on the functional status of several distinct areas of the brain, the use of the CFC assay yields more information about the efficacy of the treatment. For example, improvement in all components improves the likelihood that the immunotherapeutic will be effective.

Thirdly, the inventors have discovered that the CFC assay provides a more rapid method of evaluating the efficacy of test immunotherapeutics in improving learning and/or memory deficits. A rapid behavioral test is essential since the pharmacologically available levels of immunotherapeutic quickly decrease following their administration to the animal. After a test immunotherapeutic is administered to the animal, metabolism, adsorption, or secretion mechanisms deplete the levels of immunotherapeutic that are pharmacologically available. In exemplary embodiments, a test session is conducted when the serum levels of the test immunotherapeutic agent are at greater than 50% of the dose administered. Serum levels can be determined using methods that are well known in the art (e.g. HPLC). Preferably, the test session is conducted less than 24 hours following administration of a test immunotherapeutic agent, such that pharmacologically available levels of the agent are at their highest levels. The CFC assay requires less than 24 hours of training time for the animal, so that the effects of the immunotherapeutic can be evaluated within 24 hours of administration.

Fourthly, it is important that an assay be efficient and high-throughput in order to allow a large number of immunotherapeutic agents to be tested with a minimal demand on time and resources. The CFC assay is particularly robust and requires little laboratory equipment, less laboratory space, less physical exertion by the investigator, and only 10 minutes of test time per mouse per day. This allows the investigator the ability to evaluate many more immunotherapeutics in a shorter period of time and for considerably less expense than other methods.

The CFC tests used in the assay methods of the invention contrast distinctly with commonly used learning and memory tests such as the Morris water maze (Morris. J. Neurosci. Methods, 11: 47-60, (1984)). The Morris water maze is designed to test for procedural memories that require a relatively long period of time to be consolidated or learned. At least 3 and as many as 10 training days are required by a wild-type mouse to obtain the required criterion of escape latency from an aversive stimulus (i.e., water). Over this extended training phase, the quantity of immunotherapeutic in the bloodstream has decreased substantially. Although the water maze test has been used to demonstrate improved cognition of AD mice when actively immunized with insoluble Aβ immunogen, only a 50% reduction in the size and number of amyloid deposits was observed (Janus et al., (2000), Nature, 406: 979-82; Morgan et al, (2000), Nature, 406: 979-82) and the mice tested with this assay do not faithfully exhibit the age-related decline in cognitive performance as would be expected in human subjects (King and Arendash, Physiology & Behavior, 2002). Furthermore, the Morris water maze assay only tests contextual-dependent memories (e.g., the spatial context of a submerged platform) and the assay must be repeated with a visible platform to assess cue-dependent memory. Therefore, the water maze test is not suitably predictable of the effects of immunotherapy on cognitive deficits. Moreover, the length of time required for the number of animals per genotype that can be tested during the experiment is limited by the number of animals that can be run through the daily regimen within one day. In addition, since the same experimenter must conduct all trials, the use of this behavioral assay is exceedingly intensive in manpower requirements and radically decreases the throughput of the assay.

The CFC assay of the invention is performed in an animal chamber that provides a standard environment for the experiment (the “context”). The apparatus is a standard plastic or metal test chamber with an electrifiable grid flood, a sound and/or light source, and a calibrated shock generator. Several automated systems are commercially available including the San Diego Instruments Freeze Monitor System™ or the Motor Monitor™ system from Lafayette Instruments.

The CFC assay comprises two phases: a first “training phase” and a second “testing phase”. The training phase includes the administration of at least one “aversive stimulus” or “shock” (e.g., an electrical foot shock) that may also be concurrent with a first non-aversive sensory stimulus or “cue”. The cue may be auditory (e.g., a period of 85 db white noise), olfactory (e.g., almond or lemon extract), touch (e.g., floor cage texture), and/or visual (e.g., light flash). Typically, the cue is administered first in the absence of the shock. Shortly thereafter, a shock is co-administered with the sensory cue. This training phase may include the administration of additional sensory cues, followed by additionally co-administered shocks.

The animal's response to the shock experience is typically measured as the percentage of time during which the animal exhibits a “freezing” behavior (absence of movement except for respiration, which may also include eye blink, or change in the nictitating membrane reflex, depending on the test animal selected) relative to the total observation time. This first shock response is usually characterized on the first day of testing to determine a baseline for unconditioned fear. Typically, measurements of freezing behavior are taken at multiple time points as the experiment progresses. Scoring can be automated (e.g. photocell array) or measurements can be taken manually by an observer who is unaware of the treatment condition or the identity of the animal.

The testing phase of the CFC experiment evaluates the retention of fear conditioning performed after a certain time interval e.g. after 24 hours. The conditioned fear behavior exhibited by the animal during the testing phase (i.e., the amount of freezing) is an accurate representation of its learning and/or memory abilities. During this phase, the animal is reexposed to the environment in which it received the shock and context-dependent memory and/or cue-dependent memory is queried:

a) Context-dependent memory—the animal is tested for its ability to associate the “context” of the fear stimulus in the training session (e.g. step 7, supra), by observing the amount of freezing behavior exhibited in the same chamber used for the training phase, but in the absence of a shock. The chamber itself is representative of the context in which the conditioning occurred

An improvement in a context-dependent fear response (ie. context-dependent memory or contextual memory) is determined by comparing the fear response of the animal to the context-dependent fear stimulus with a fear response of the model animal in the absence of a context-dependent fear stimulus. To quantitate the contextual memory, the percentage of freezing observed during the training phase may be subtracted from the percentage of freezing observed during the testing phase.

b) Cue-dependent memory the animal may also optionally be tested for its ability to associate a “cue” associated with the fear stimulus in the training session by carrying out additional tests (e.g. steps 8-10, supra). The animal is removed from the chamber in which the training phase was conducted and it is placed in an altered environment or context (e.g., a cage of different shape, texture, or lighting). Freezing behavior in the novel environment is scored (e.g. see step 9, supra). Following its exposure to the novel environment or context, the animal is given the same non-averse stimulus or “cue” (ie. an auditory cue) that was given during the training session, and the freezing behavior is noted. Freezing behavior in the altered environment with a cue is compared with that observed in the altered environment without the cue, to determine the degree of cue-dependent memory. Alternatively, the improvement in the fear response is determined by comparing the fear response of the animal to the cue-dependent fear stimulus with a fear response of the model animal in the absence of a cue-dependent fear stimulus.

In certain embodiments, the CFC assay may be altered to suit the purposes of the experimenter. In one exemplary embodiment, steps 1-3 of the above exemplary CFC assay may be repeated one or more times during the training session. In another embodiment, the training session may be repeated over one or more training days before initiating a new testing session. It will be appreciate that the timing of each phase of the above exemplary assay may also be varied (e.g. by 5, 10, 15, 30, 60, or 120 seconds).

### III) MODEL ANIMALS

The methods of the invention may be employed using model animals that exhibit prominent symptoms and/or pathology that are characteristic of a dementia disorder, particularly an amyloidogenic disorder such as Alzheimer's.

In certain embodiments, model animals are evaluated using the methods of the invention at an age when they display symptoms of the disease (e.g. memory deficits), but lack disease pathology (e.g. plaque formation). In exemplary embodiments, model animals are evaluated using the methods of the invention when they are approximately 10 weeks of age or older, more preferably when they are approximately 20 weeks of age or older. In particular, approximately 20 week old transgenic AD mice have particularly prominent contextual memory deficits, and are accordingly preferred model animals for the methods of the invention.

In other embodiments, model animals are evaluated using the methods of the invention at an age when they display both the symptoms of the disease (e.g. memory deficits) and the disease pathology. In exemplary embodiments, model animals are evaluated when they are approximately 10 months of age or older, or when they are greater that 15 months of age or older. In a preferred embodiment, approximately 18-20 month old transgenic AD mice are evaluated using the methods of the invention. 18-20 month old transgenic AD mice are relatively aged animals and are likely to have dense plaque formations in their brains, as well prominent memory deficits.

Model animals may be created by selective inbreeding for a desired symptom or pathology or they may be genetically engineered using transgenic techniques that are well-known in the art. Transgenic animals may contain a genetic alteration (e.g. a genetic mutation, gene disruption) in a gene that is associated with the dementia disorder, leading to aberrant expression or function of the targeted gene. Alternatively, the model animal can be created using chemical compounds or surgical techniques that ablate or otherwise interfere with the function of an anatomical brain region that is associated with the characteristic symptoms or pathology of the dementia disorder.

a) Transgenic Animal Models:

Several mouse models have been used successfully to determine the significance of amyloid plaques in Alzheimer's (Games et al., supra, Johnson-Wood et al., Proc. Natl. Acad. Sci. USA 94:1550 (1997)).

i) PDAPP: PDAPP mice express a mutant form of human APP (APP^{V717F}) and develop Alzheimer's disease at a young age. (Bard, et al. (2000) Nature Medicine 6:916-919; Johnson-Wood, et al. (1997), Proc. Natl. Acad. Sci. USA, 94: 1550-5; Masliah E, et al. (1996) J Neurosci. 15;16(18):5795-8 11; Games et al., (1995) Nature, 373: 523-7)). They also develop age-dependent object recognition memory impairments (Dodart, et al. (1999) Behav. Neurosci. 113: 982-90). PDAPP transgenic mice, are injected with the long form of Aβ, they display both a decrease in the progression of Alzheimer's and an increase in antibody titers to the Aβ peptide (Schenk et al., Nature 400: 173 (1999)).

ii) Tg2576: Tg2576 mice overexpress the “Swedish” mutation of human APP₆₉₅ (hAPPswe; Tg2576) and develop age-dependent neural defects including increased, amyloid plaque deposition, alterations in synaptic function, and deficits in cognitive function, particularly memory deficits in hippocampal-dependent learning tasks (Chapman et al. (1999) Nat. Neurosci. 2: 271-6; Hsiao et al. (1996) Science 274:99-102). Tg2576 mice do not typically have evidence of plaque deposition until they are 18 months of age. Plaques are particularly concentrated in certain regions of the brain such us the cortex, hippocampus, and amygdala (Chapman et al, (2001) Trends Genet. 17: 254-261).

The onset of impaired memory in Tg2576 mice begins as early as 5 months of age. Therefore, it is desirous that an immunotherapeutic agent is administered Tg2576 mice between 5 and 8 months of age and prior to training with CFC assay. In particularly preferred embodiments, Tg2576 mice are evaluated in a CFC when they are greater than 10 weeks of age, more preferably when they are approximately 20 weeks of age. In particular, 20 week old mice have particularly prominent contextual memory deficit, and are accordingly preferred model animals for the methods of the invention. Aβ-dependent amyloid effect is determined by comparing the response of Tg2576 with that of wild-type mice. Aβ-dependent amyloid effect is one in which the attenuating effect of the agent on the mutant phenotype is substantially greater than the attenuating effect of the agent on the wild-type phenotype.

iii) TgAPP22: TgAPP22 mice contain the V717F mutation in addition to the Swedish mutation (Sturchler-Pierrat et al. (1997), Proc. Natl. Acad. Sci. USA 94: 6483-92).

iv) TgAPP/LD12: TgAP/LD/2 mice contain the V717I mutation in APP₆₉₅ (Moechars et al., (1999), J. Biol. Chem. 274: 6483-92.

v) PSEN-1 A246E: This mutant mouse expresses a mutant form of the human presenilin-1 (PSEN-1) gene (where glutamic acid replaces alanine at position 246) resulting in enhanced deposition of Aβ (Duff, et al. (1996) Nature 383, 710-713).

vi) PSEN-1 DeltaE9: This mutant mouse expresses a mutant form of PSEN-1 where exon 9 is mispliced (Duff, et al. (1996) Nature 383, 710-713).

vii) Tg2576+PSEN-1: This mouse is a double mutant containing the Swedish mutation in addition to PSEN-1 (Holcomb et al., (1998) Nat. Med. 4: 97-100.

viii) TgHu/MoAPP A246E+PSEN-1: This double transgenic mouse expresses a chimeric mouse/human APPswe transgene in conjunction with a PSEN-1 transgene bearing the A246E mutation (Borchelt et al, (1997) Neuron 19: 939-45). Histological examination reveals that amyloid plaques appear in the brain tissue of these mice at 9 months of age, much earlier than in mice bearing only the APPswe transgene.

ix) TgHu/MoAPP DeltaE9+PSEN-1: This double mutant expresses the Delta E9 PSEN-1 mutant in conjunction with the chimeric mouse/human APPswe transgene and exhibits amyloid deposits at only 6 months of age.

x) TgCDNR8: TgCDNR8 overexpress a mutant form of APP (McLaurin et al. (2002) Nat. Med. 8: 1263-9).

xi) PSAPP: PSAPP is a doubly transgenic mouse that overexpresses mutant forms of both APP (e.g. Swedish-type mutant APP (hAPpswe)) and PSEN-1 (e.g. mutant human PS1_{M146L}) (see Holcomb et al., (1998) Nat. Med, 4: 97-110). PSAPP mouse models exhibit age-related development of amyloid plaques that is similar to that observed in AD (Kumar-Singh et al., Am J Pathol. (2005), 167(2):527-43). Deposition of Aβ in the frontal cortex and hippocampus of PSAPP mice as early as 3 months of age progresses to cover substantial portions of these areas of the brain at 12 months (Takachi et al., Am J Pathol. (2000), 157(l):331-9; McGowan et al., Neurobiol Dis. (1999), 6(4):231-44). PSAPP mice can be evaluated in a CFC when they are greater than 10 months of age, for example, when they are approximately 20 months of age. In particular, 20 month old PSAPP mice have particularly prominent contextual memory deficit and dense accumulation of plaque, and are accordingly exemplary model animals for the methods of the invention.

xii) 3xTg-AD: 3xTg-AD is a triple transgenic mouse model harboring three mutant genes: beta-amyloid precursor protein (betaAPPSwe), presenilin-1 (PS1M146V), and tauP301L. The 3xTg-AD mice progressively develop Abeta and tau pathology, with a temporal- and regional-specific profile that closely mimics their development in the human AD brain (Oddo et al., Neurobiol Aging. (2003), 24(8):1063-70; Oddo et al., Neuron. (2003), 39(3):409-21).

Other genetically altered transgenic models of Alzheimer's disease are described in Masliah E and Rockenstein E. (2000) J Neural Transm Suppl. 59:175-83.

b) Lesion Animal Models:

Non-human animal models with wild-type alleles of Alzheimer's related proteins can be used to test the efficacy of candidate immunotherapeutic agents by introducing lesions in anatomical regions of the brain that are critical for memory consolidation.

In one embodiment, lesions can be introduced to the formix structure of the brain, such that formix-mediated signaling to the hippocampus is at least partially disrupted. Animals with formix lesions learn inhibitory avoidance and display memory at control levels for up to 6 hours, but by 24 hours they exhibit amnesia (Taubenfeld et al. (1999) Nat. Neurosci., 2: 309). Other regions of the brain that are critical for learning and memory can also be specifically disrupted (e.g. hippocampus, amygdale, perirhinal cortex, medial septal nucleus, locus coeruleus, mammalary bodies) using techniques that are well known in the art.

Permanent brain lesions in the mammal can be produced by mechanical disruption, e.g. with surgical ablation or electrolytic treatment, or by chemical ablation with a neurotoxin, e.g. tetradotoxin. Surgical ablation techniques can include stereotactic ablation, axotomization, transection, or aspiration. Alternatively, a temporary brain lesion can be introduced by chemical agents that temporarily arrest neurological activity in these regions of the brain e.g. local anaesthetics such as lidocaine.

In certain preferred embodiments, the brain lesion can be generated by selective disruption of specific neurons that are classified on the basis of neurotransmitter release:

i) Serotonergic neurons can be specifically ablated by injection of methamphetamines ( e.g. methylenedioxyamphetamine (MDA), d-methamphetamine (d-MA), methylenedioxymethamphetamine (MDMA), 5,7-dihydroxytryptamine (5,7-DHT);

ii) Cholinergic neurons can be specifically ablated by injection of atropine or 1921gG-saporin;

iii) Dopaminergic neurons can be specifically ablated by injection of 6-OHDA or ibotenic acid.

### IV) CANDIDATE IMMUNOTHERAPEUTICS FOR TESTING WITH THE CFC ASSAY

Candidate immunotherapeutic agents useful for testing with the methods of the invention include immunogens which are administered as an active immunotherapies, as well as antibodies or functional or antigen binding fragments thereof, which are administered as passive immunotherapeutics.

In one embodiment of the invention, the test immunotherapeutic is capable of binding insoluble Aβ deposited in plaques to decrease the size and density of amyloid plaques. In another embodiment, the test immunotherapeutic is capable of capturing soluble Aβ, including monomeric soluble as well as oligomeric soluble Aβ polypeptide (e.g., soluble Aβ dimers, trimers, and the like), and preventing accumulation of Aβ and/or promoting removal of Aβ from the CNS.

In a particularly preferred embodiment, the test immunotherapeutics are capable of binding to both soluble and insoluble Aβ peptides or fragments thereof, and are capable of preventing the formation of additional amyloid plaque while also decreasing the size and density of existing amyloid plaques.

a) Active Immunotherapeutics

In certain aspects of the methods of the present invention, an immunotherapeutic agent to be tested for therapeutic efficacy (a test immunotherapeutic agent) is an active immunotherapeutic consisting of a protein, peptide, carbohydrate, lipid, or nucleotide immunogen that accumulates during the progression of a dementia disorder. In particular embodiments, the methods of the present invention utilize immunogenic preparations of amyloid proteins such as an Aβ peptide or fragments thereof. In an exemplary embodiment, the peptide immunogen comprises from about 1 to about 42 amino acid residues of the human Aβ₁₋₄₂ peptide. In another exemplary embodiment, the peptide immunogen comprises from about 4 to about 10 amino acid residues from the human Aβ₁₋₄₂ peptide.

Candidate immunogens may comprise either soluble or insoluble forms of Aβ peptide or fragments or portions thereof. In some preferred embodiments, the candidate immunogen is or comprises an epitope or antigenic determinant from any soluble Aβ peptide, including a monomeric soluble and/or oligomeric soluble Aβ polypeptide (e.g., soluble Aβ dimers, trimers, and the like). Some exemplary epitopes or antigenic determinants are located within the N-terminus of the Aβ peptide and include residues within amino acids 1-10, 1-5, or 5-10 of Aβ, preferably from residues 1-3, 1-4, 1-5, 1-6, 1-7 or 3-7 of Aβ42. Other exemplary epitopes or antigenic determinants start at resides 1-3 and end at residues 7-11 of Aβ. Other exemplary epitopes or antigenic determinants comprise residues 10-15, 15-20, 25-30, 10-20, 20-30, or 10-25 of Aβ. Additional exemplary epitopes or antigenic determinants include residues 2-4, 5, 6, 7 or 8 of Aβ, residues 3-5, 6, 7, 8 or 9 of Aβ, or residues 4-7, 8, 9 or 10 of Aβ42. Such epitopes can be referred to as N-terminal epitopes. Additional exemplary epitopes or antigenic determinants include residues 19-22, 23 or 24 of Aβ42. Other exemplary epitopes or antigenic determinants include residues 10-18, 16-24, 18-21 and 33-42 of Aβ42. Additional exemplary epitopes or antigenic determinants include residues 16-21, 22, 23 or 24 of Aβ42. Such epitopes can be referred to as central epitopes. Additional exemplary epitopes or antigenic determinants include residues 33-40 or 33-42 of Aβ. Such epitopes can be referred to as C-terminal epitopes. In some embodiments, the immunogen is or comprises Aβ₁₃₋₂₈, Aβ₁₇₋₂₈, Aβ₂₅₋₃₅, Aβ₃₅₋₄₀, or Aβ₃₅₋₄₂.

When administered to an animal, the candidate immunogen is expected to induce an immune response, e.g. antibody production, against one or more antigenic determinants or epitopes within the immunogen. For example, a test immunogen can be taken up by antigen-presenting cells and antigenic fragments of the test immunogen are presented to helper T cells which induce B-cell production of antibodies by B cells that specifically bind to the same antigenic determinant or epitope on endogenous Aβ peptides. Some such antibodies may bind specifically to the aggregated form of Aβ without binding to the soluble form. Some may bind specifically to the soluble form without binding to the aggregated form. Some may bind to both aggregated and soluble forms. In a preferred embodiment, the Aβ immunogen is capable of inducing the production of antibodies against the main functional site of the Aβ peptide that exhibit high cross-reactivity both to soluble and aggregated forms of Aβ peptide.

In an alternative embodiment, Aβ peptides or fragments thereof, may be coupled at their carboxy- or amino-termini to carrier proteins (e.g. human serum albumin, keyhold limpet hemocyanin, immunoglobulin molecules, thyroglobulin, ovalbumin, influenza hemagluttinin, PAN-DR binding peptide (PADRE polypeptide), malaria circumsporozite (CS) protein, hepatitis B surface antigen (HBₛAg₁₉₋₂₈), Heat Shock Protein (HSP) 65, a polypeptide from Mycobacteriarm tuberculosis [Bacillus Calmette-Guerin (BCG)], tetanus toxin or toxoids, cholera toxin or toxoids, cholera toxin mutants with reduced toxicity, diphtheria toxin or toxoids, CRM₁₉₇ protein that is cross-reactive with diphtheria toxin, recombinant Streptococcal C5a peptidase, Streptococcus pyogenes ORF1224, Streptococcus pyogenes ORF1664, Streptococcus pyogenes ORF2452, Chlamydia pneumoniae ORF T367, Chlamydia pneumoniae ORF T858, HIV gp120 T1, microbial surface components recognizing adhesive matrix molecules (MSCRAMMS), a growth factor, cytokine, chemokine or hormone, which stimulates or enhances immune responses such as of IL-1, IL-2, γ-interferon, IL-10, GM-CSF, MIP-1α, and RANTES, etc.) using recombinant DNA or chemical coupling techniques, in order to form a conjugate protein or polypeptide with enhanced immunogenicity.

Aβ peptide immunogens can be derived from a variety of sources, for example, tissues, cell lines, or body fluids (e.g. sera or cerebrospinal fluid). For example, an Aβ immunogen can be derived from APP-expressing cells such as Chinese hamster ovary (CHO) cells stably transfected with APP_{717V→F} (referred to as 7PA2 cells) and cultured in Dulbecco's modified Eagle's medium (DMEM) with 10% fetal bovine serum, as described, for example, in Walsh et al., (2002), Nature, 416, pp 535-539. An Aβ preparation can be derived from tissue sources using methods previously described (see, e.g., Johnson-Wood et al., (1997), Proc. Natl. Acad. Sci. USA 94:1550).

Alternatively, the Aβ immunogen can be synthesized using methods which are well known to those in the art. See, for example, Fields et al., Synthetic Peptides: A User's Guide, ed. Grant, W.H. Freeman & Co., New York, N.Y., 1992, p 77). Hence, peptides can be synthesized using the automated Merrifield techniques of solid phase synthesis with the α-amino group protected by either t-Boc or F-moc chemistry using side chain protected amino acids on, for-example, an Applied Biosystems Peptide Synthesizer Model 430A or 431.

Longer peptide immunogens can be synthesized using well known recombinant DNA techniques. For example, a polynucleotide encoding the peptide or fusion peptide can be synthesized or molecularly cloned and inserted in a suitable expression vector for the transfection and heterologous expression by a suitable host cell.

Immunogens may optionally be formulated into pharmaceutically acceptable formulations by the addition of one or more adjuvants. The term “adjuvant” refers to a compound that when administered in conjunction with an immunogen augments the immune response to the immunogen, but when administered alone does not generate an immune response. Adjuvants can augment an immune response by several mechanisms including lymphocyte recruitment, stimulation of B and/or T cells, and stimulation of macrophages. Several types of adjuvant can be used as described below. Exemplary adjuvants include, but are not limited to, calcium salts such as calcium phosphate, Complete Freund's Adjuvant (CFA), Incomplete Freund's Adjuvant (IFA), alums (e.g. aluminum hydroxide, aluminum phosphate, or aluminum sulfate), Amphigen, Avridine, L121/squaline, D-lactide polylactide/glycoside, pluronic acids, polyols, muramyl dipeptide, killed Bordetella, Mycobacterium tuberculosis, bacterial lipopolysaccharides, synthetic polynucleotides such as oligonucleotides containing a CpG motif (see U.S. Pat. No. 6,207,646, which is incorporated by reference), a pertussis toxin (PT), or an E. coli heat-labile toxin (LT), particularly LT-K63, LT-R72, PT-K9/G129 (see, e.g., International Patent Publication Nos. WO 93/13302 and WO 92/19265, which are incorporated herein by reference).

Also useful as adjuvants are cholera toxins and mutants thereof, including those described in published International Patent Application No. WO 00/18434 (wherein the glutamic acid at amino acid position 29 is replaced by another amino acid (other than aspartic acid, preferably a histidine). Similar CT toxins or mutants are described in published International Patent Application No. WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid; and/or an amino acid is inserted at amino acid position 49; and/or two amino acids are inserted at amino acid positions 35 and 36).

A number of cytokines or lymphokines have been shown to have immune modulating activity, and thus may also be used as adjuvants, including, but not limited to, the interleukins (e.g., IL-1α, IL-1β, IL-2, IL-4, IL-5, IL-6, IL-8, IL-10, IL-12, IL-13, IL-14, IL-15, IL-16, IL-17, IL-18, and their mutant forms (see, e.g., U.S. Pat. No. 5,723,127), the interferons-α, β, and γ, macrophage colony stimulating factor (M-CSF), granulocyte-macrophage colony stimulating factor (G-CSF, see, e.g., U.S. Pat. No. 5,078,996), and tumor necrosis factors (TNFα and TNFβ). Still other adjuvants useful in this invention include a chemokine (e.g. MCP-1, MIP-1α, MIP-1β, and RANTES). Adhesion molecules such as selectin (e.g. L-selectin, P-selectin, and E-selectin) may be useful as adjuvants. Still other useful adjuvants include without limitation, a mucin-like molecule (e.g., CD34, GlyCAM-1, and MadCAM-1), a member of the integrin family (e.g. LFA-1, VLA-1, Mac-1 and p150.95), a member of the immunoglobulin superfamily (e.g. PECAM, CD, LFA-3, and ICAMs such as ICAM-1, ICAM-2, and ICAM-3), co-stimulatory molecules such as CD40 and CD40L, growth factors (e.g., vascular growth factor, nerve growth factor, fibroblast growth factor, epidermal growth factor, B7.2, PDGF, BL-1, and vascular endothelial growth factor), receptor molecules (e.g. Fas, TNF receptor, Flt, Apo-1, p55, WSL-1, DR3, TRAMP, Apo-3, AIR, LARD, NGRF, DR4, DR5, KILLER, TRAIL-R2, and DR6), and caspases (e.g. ICE). See, also International Patent Publication Nos. WO98/17799 and WO99/43839, incorporated herein by reference.

Still other suitable adjuvants used to enhance an immune response include, without limitation, liposyns, saponins (e.g. Stimulon™ QS-21, Antigenics, Framingham, Mass., described in U.S. Pat. No. 5,057,540, and particles generated therefrom such as ISCOMS (immunostimulating complexes), glycolipid analogues (e.g. N-glycosylamides, N-glycosylureas, and N-glycosylcarbamates, see U.S. Pat. No.4, 855,283), squalene, L121, 3-O-deacylated monophosphoryl lipid A (MPL™, Corixa, Hamilton, Mont., described in U.S. Pat. No. 4,912,094, which is hereby incorporated by reference), polysorbate 80, QS21, Montanide, ISA51, ISA35, ISA206, and ISA720, as well as other efficacious adjuvants that are well known in the art, such as oil in water emulsions (e.g. squalene or peanut oil), optionally in combination with immune stimulants such as monophosphoryl lipid A, pluronic polymers, and killed mycobacteria. Also suitable for use as adjuvants are synthetic lipid A analogs or aminoalkyl glucosamine phosphate compounds (AGP), or derivatives or analogs thereof, which are available from Corixa (Hamilton, Mont.), and which are describe in U.S. Pat. No. 6,113,918, which is hereby incorporated by reference. One such AGP is 2-[(R)-3-Tetradecanoyloxytetradecancylamino]ethyl 2-Deoxy-4-O-phosphono-3-O-[(S)-3-tetradecanoyoxytetradecanoyl]-2-[(R)-3-tetradecanoyloxytetradecanoyl-amino]-b-D-glycopyranoside, which is also known as 529 (formerly known as RC529). This adjuvant is formulated as an aqueous form or as a stable emulsion. An exemplary immunogenic formulation comprises 5-10000 μg of peptide immunogen formulated as a water-in-oil emulsion in a pharmaceutically acceptable adjuvant.

Candidate immunogens can be administered to a non-human animal model via any conventional route, such as subcutaneous, oral, intravenous, intramuscular, parenteral, intranasal, or enteral route. In a preferred embodiment, the immunogen is administered by an intramuscular route.

Candidate immunogens can be administered to a non-human animal model in a single dose or in multiple doses. Each additional dose in commonly referred to as a booster dose. In a preferred embodiment, the three doses of immunogen are administered to the non-human animal. In another preferred embodiment, the initial dose is administered in Complete Freund's Adjuvant, and each booster dose is administered in Incomplete Freund's Adjuvant.

Candidate immunogens can be administered to a non-human animal model during, prior to, or following testing in a fear conditioning assay. In one embodiment, a single dose of immunogen is administered to a non-human animal model prior to testing in a fear conditioning assay. In a more preferred embodiment, the single dose of immunogen is administered to a non-human animal model, at least one week prior to testing in a fear conditioning assay. In another embodiment, multiple doses of immunogen are administered to a non-human animal model, with the last dose administered at least one week prior to testing in a fear conditioning assay. In a more preferred embodiment, multiple doses of immunogen are administered at 9, 5, and 1 week prior to testing in a fear conditioning assay.

Candidate immunogens can be administered at a dosage of about 0.25 μg to about 500 μg of immunogen per kg of body weight. When delivered in multiple doses, the effective amount may be conveniently divided per dosage unit. For example, an initial dose, e.g. 0.0025-0.5 mg per kg of body weight, preferably 1-50 μg per kg of body weight, is to be administered by injection, followed by booster doses of a similar amount. Dosage will depend on the age, weight, and general health of the subject as is well known in the art.

b) Passive Immunotherapeutic Agents

Passive immunotherapeutic agents include antibodies that specifically bind to proteins or peptides that accumulate during the progression of a dementia disorder. This approach does not require any type of immunological response from the host and, as such, has the potential to be useful in animals that might not otherwise generate an immune response to Aβ peptide.

In particular, the methods of the present invention utilize candidate antibodies that bind components of amyloid deposits such as an Aβ peptide or fragments thereof. Preferred candidate antibodies are monoclonal antibodies. Some candidate antibodies bind specifically to the aggregated form of Aβ without binding to the soluble form. Some bind specifically to the soluble form without binding to the aggregated form. Some bind to both aggregated and soluble forms. Some candidate antibodies bind Aβ in plaques. Some candidate antibodies can cross the blood-brain barrier. Some candidate antibodies can reduce amyloid burden in a subject. Some candidate antibodies can reduce neuritic dystrophy in a subject. Some candidate antibodies can maintain synaptic architecture (e.g., synaptophysin).

In a preferred embodiment, candidate antibodies perform well (i.e., reverse cognitive deficits and/or reverse cognitive impairments) in the CFC assays of the invention. Exemplary antibodies perform well in the CFC assays of the invention over a prolonged period (e.g., for at least 5 days or 10 days). Test antibodies may further bind soluble Aβ peptide including monomeric soluble and/or oligomeric soluble Aβ polypeptide (e.g., soluble Aβ dimers, trimers, and the like).

Preferred test antibodies bind to Aβ with a binding affinity greater than (or equal to) about 10⁶, 10⁷, 10⁸, 10⁹, or 10¹⁰ M⁻¹ (including affinities intermediate of these values).

Candidate antibodies also include those antibodies which are capable of binding and/or clearing soluble Aβ in the CNS or brain of a subject. Exemplary candidate antibodies also include those antibodies which are capable of capturing soluble Aβ, e.g., in the bloodstream of a subject. Preferred candidate antibodies are capable of rapidly improving cognition in a subject, e.g., via clearance and/or capture of soluble Aβ. Preferred candidate antibodies of the invention include murine and humanized versions of anti-Aβ antibodies which bind to epitopes in the N-terminus, central region, or C-terminus of Aβ peptide. Exemplary N-terminal anti-Aβ antibodies include 3D6 (which binds to a region from approximately residue 1 to approximately residue 5), 2H3 (which binds to a region from approximately residue 2 to approximately residue 7), 10D5 (which binds to a region from approximately residue 3 to approximately residue 6), and 6C6, 12A11, 12B4, 3A3, and 2C1 (which bind to a region from approximately residue 3 to approximately residue 7). Exemplary central region anti-Aβ antibodies include 9G8 (which binds to a region from approximately residue 16 to approximately residue 21), R9C5 (which binds to a region from approximately residue 16 to approximately residue 22), 1C2 (which binds to a region from approximately residue 16 to approximately residue 23), 266 (which binds to a region from approximately residue 16 to approximately residue 24), 22D12 (which binds to a region from approximately residue 19 to approximately residue 21), 15C11 and 6H9 (which bind to a region from approximately residue 19 to approximately residue 22), 3D8, 2B1, and 5A11 (which bind to a region from approximately residue 19 to approximately residue 23), and 3F8 (which binds to a region from approximately residue 19 to approximately residue 24). Exemplary C-terminal anti-Aβ antibodies include 2G3 and 14C2 (which bind to a region from approximately residue 30 to approximately residue 40) and 21F12 and 16C11 (which bind to a region from approximately residue 30 to approximately residue 42).

Epitope specificity of a candidate antibody can be determined, for example, by forming a phage display library in which different members display different subsequences of Aβ. The phage display library is then selected for members specifically binding to an antibody under test. A family of sequences is isolated. Typically, such a family contains a common core sequence, and varying lengths of flanking sequences in different members. The shortest core sequence showing specific binding to the antibody defines the epitope bound by the antibody. Candidate antibodies can also be tested for epitope specificity in a competition assay with an antibody whose epitope specificity has already been determined.

Epitope specificity can also be determined using replacement NET (rNET) analysis. The rNET epitope map assay provides information about the contribution of individual residues within the epitope to the overall binding activity of the antibody. rNET analysis uses synthesized systematic single substituted peptide analogs. Binding of an antibody being tested is determined against native peptide (native antigen) and against 19 alternative “single substituted” peptides, each peptide being substituted at a first position with one of 19 non-native amino acids for that position. A profile is generated reflecting the effect of substitution at that position with the various non-native residues. Profiles are likewise generated at successive positions along the antigenic peptide. The combined profile, or epitope map, (reflecting substitution at each position with all 19 non-native residues) can then be compared to a map similarly generated for a second antibody. Substantially similar or identical maps indicate that antibodies being compared have the same or similar epitope specificity

The candidate Aβ antibodies used for passive administration in a non-human animal model of AD are also produced in a non-human mammal, e.g., murine, guinea pig, primate, rabbit or rat, by immunizing the animal with Aβ. A longer polypeptide comprising Aβ or an immunogenic fragment of Aβ or anti-idiotypic antibodies to an antibody to Aβ can also be used. See Harlow & Lane, supra, incorporated by reference for all purposes). Such a polypeptide can be obtained from a natural source, by peptide synthesis or by recombinant expression. Optionally, Aβ, or an immunogenic fragment thereof, can be administered fused or otherwise complexed with a carrier protein, as described below. Optionally, the Aβ immunogen can be administered with an adjuvant. Complete Freund's adjuvant followed by incomplete adjuvant is preferred for immunization of laboratory animals.

Mice, rabbits or guinea pigs are typically used for making polyclonal antibodies. Exemplary preparation of polyclonal antibodies, e.g., for passive protection, can be performed as follows. 125 non-transgenic mice are immunized with 100 μg Aβ1-42, plus CFA/IFA adjuvant, and euthanized at 4-5 months. Blood is collected from immunized mice. IgG is separated from other blood components. An antibody specific for the immunogen may be partially purified by affinity chromatography. An average of about 0.5-1 mg of immunogen-specific antibody is obtained per mouse, giving a total of 60-120 mg.

Mice are typically used for making monoclonal antibodies. Monoclonals can be prepared against a fragment by injecting the fragment or longer form of Aβ into a mouse, preparing hybridomas and screening the hybridomas for an antibody that specifically binds to Aβ. Optionally, antibodies are screened for binding to a specific region or desired fragment of Aβ without binding to other nonoverlapping fragments of Aβ. The latter screening can be accomplished by determining binding of an antibody to a collection of deletion mutants of an Aβ peptide and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to Aβ. If the test and reference antibodies compete, then they bind to the same epitope or epitopes sufficiently proximal such that binding of one antibody interferes with binding of the other. The preferred isotype for such antibodies is mouse isotype IgG2a or equivalent isotype in other species. Mouse isotype IgG2a is the equivalent of human isotype IgG1 (e.g., human IgG1).

The antibodies identified by the methods of the invention and used for passive immunization or immunotherapy of human subjects with dementia disorders can be human, humanized, chimeric or nonhuman antibodies, or fragments thereof (e.g., antigen binding fragments) and can be monoclonal or polyclonal, as described herein.

Exemplary antibodies for use in passive immunotherapy (e.g., humanized immunoglobulins) are preferably directed to specific epitopes within Aβ peptide that are associated with amyloid deposits in the brain of a patient. In one embodiment the therapeutic antibodies specifically bind to Aβ peptide without binding to full-length amyloid precursor protein (APP). In yet another aspect, the isotype of the therapeutic antibody is human IgG1. In yet another embodiment, the invention features administering candidate antibodies that bind to and/or capture soluble Aβ. In yet another aspect, the invention features administering antibodies that bind to an amyloid deposit in the patient and induce a clearing response against the amyloid deposit. For example, such a clearing response can be effected by Fc receptor mediated phagocytosis. Such a clearing response can be engineered into an antibody, for example, by including an Fc receptor binding domain (e.g., an IgG2a constant region).

In one embodiment, the non-human antibody which is determined as being efficacious in the non-human animal model is “humanized” before it is administered to the patient. Humanization of a candidate antibody requires an analysis of the appropriate mouse CDR components which substituted into a suitable a human variable domain framework is most likely to result in retention of the correct spatial orientation. This is particularly likely if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993) and Carter et al., WO 92/22653.

Having identified the CDRs of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined by computer modeling the amino acid sequence similarity of the candidate antibody with chains or domains of solved three-dimensional structures, and the chains or domains showing the greatest sequence similarity is/are selected as starting points for construction of the molecular model. The selection of amino acid residues for substitution can also be determined, in part, by examination of the characteristics of the amino acids at particular locations, or empirical observation of the effects of substitution or mutagenesis of particular amino acids. Amino acids that are capable of interacting with amino acids in the CDRs, may additionally be identified by calculating the solvent accessible surface area of each framework amino acid to determine if it is at least partly blocked by the CDRs, and therefore making contact with the CDRs (e.g., Connolly, J. Appl. Cryst. 16:548 (1983) and Lee and Richards, J. Mol. Biol. 55:379 (1971), both of which are incorporated herein by reference). Additional candidates for substitution are acceptor human framework amino acids that are unusual or “rare” for a human immunoglobulin at that position. These amino acids can be substituted with amino acids from the equivalent position of the mouse donor antibody or from the equivalent positions of more typical human immunoglobulins.

Additional candidates for substitution are acceptor human framework amino acids that would be identified as part of a CDR region under the alternative definition proposed by Chothia et al., supra. Additional candidates for substitution are acceptor human framework amino acids that would be identified as part of a CDR region under the AbM and/or contact definitions.

Additional candidates for substitution are acceptor framework residues that correspond to a rare or unusual donor framework residue. Rare or unusual donor framework residues are those that are rare or unusual (as defined herein) for murine antibodies at that position. Unusual residues that are predicted to affect binding are retained, whereas residues predicted to be unimportant for binding can be substituted.

Additional candidates for substitution are non-germline residues occurring in an acceptor framework region. For example, when an acceptor antibody chain (i.e., a human antibody chain sharing significant sequence identity with the donor antibody chain) is aligned to a germline antibody chain (likewise sharing significant sequence identity with the donor chain), residues not matching between acceptor chain framework and the germline chain framework can be substituted with corresponding residues from the germline sequence.

In general, one or more of the amino acids fulfilling the above criteria is substituted. In some embodiments, all or most of the amino acids fulfilling the above criteria are substituted. Occasionally, there is some ambiguity about whether a particular amino acid meets the above criteria, and alternative variant immunoglobulins are produced, one of which has that particular substitution, the other of which does not. Alternative variant immunoglobulins so produced can be tested in any of the assays described herein for the desired activity, and the preferred immunoglobulin selected.

Having conceptually selected the CDR and framework components of humanized immunoglobulins, a variety of methods are available for producing such immunoglobulins. In general, one or more of the murine complementarity determining regions (CDR) of the heavy and/or light chain of the antibody can be humanized, for example, placed in the context of one or more human framework regions, using primer-based polymerase chain reaction (PCR). Briefly, primers are designed which are capable of annealing to target murine CDR region(s) which also contain sequence which overlaps and can anneal with a human framework region. Accordingly, under appropriate conditions, the primers can amplify a murine CDR from a murine antibody template nucleic acid and add to the amplified template a portion of a human framework sequence. Similarly, primers can be designed which are capable of annealing to a target human framework region(s) where a PCR reaction using these primers results in an amplified human framework region(s). When each amplification product is then denatured, combined, and annealed to the other product, the murine CDR region, having overlapping human framework sequence with the amplified human framework sequence, can be genetically linked. Accordingly, in one or more such reactions, one or more murine CDR regions can be genetically linked to intervening human framework regions.

The variable segments of antibodies produced as described supra (e.g., the heavy and light chain variable regions of humanized antibodies) are typically linked to at least a portion of an immunoglobulin constant region (Fc region), typically that of a human immunoglobulin. Human constant region DNA sequences can be isolated in accordance with well known procedures from a variety of human cells, but preferably immortalized B cells (see Kabat et al., supra, and Liu et al., WO87/02671) (each of which is incorporated by reference in its entirety for all purposes). When it is desired that the humanized antibody exhibit cytotoxic activity, the constant domain is usually a complement fixing constant domain and the class is typically IgG1. Human isotype IgG1 is preferred. Light chain constant regions can be lambda or kappa. The humanized antibody may comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

For the above-described antibodies comprising a constant region (Fc region), it may also be desirable to alter the effector function of the molecule. Generally, the effector function of an antibody resides in the constant or Fc region of the molecule which can mediate binding to various effector molecules, e.g., complement proteins or Fc receptors. The binding of complement to the Fc region is important, for example, in the opsonization and lysis of cell pathogens and the activation of inflammatory responses. The binding of antibody to Fc receptors, for example, on the surface of effector cells can trigger a number of important and diverse biological responses including, for example, engulfment and destruction of antibody-coated pathogens or particles, clearance of immune complexes, lysis of antibody-coated target cells by killer cells (i.e., antibody-dependent cell-mediated cytotoxicity, or ADCC), release of inflammatory mediators, placental transfer of antibodies, and control of immunoglobulin production.

Accordingly, depending on a particular therapeutic or diagnostic application, the above-mentioned immune functions, or only selected immune functions, may be desirable. By altering the Fc region of the antibody, various aspects of the effector function of the molecule, including enhancing or suppressing various reactions of the immune system, with beneficial effects in diagnosis and therapy, are achieved.

Antibodies can be produced which react only with certain types of Fc receptors, for example, antibodies can be modified to bind to only certain Fc receptors, or if desired, lack Fc receptor binding entirely, by deletion or alteration of the Fc receptor binding site located in the Fc region of the antibody.

As a further example, the lytic properties of IgG antibodies following binding of the Cl component of complement can be altered. The first component of the complement system, Cl, comprises three proteins known as Clq, Clr and Cls which bind tightly together. It has been shown that Clq is responsible for binding of the three protein complex to an antibody. Accordingly, the Clq binding activity of an antibody can be altered by providing an antibody with an altered Fc region in order to reduce or abolish specific Clq-binding.

The antibodies of the present invention can be administered to a non-human animal model via any conventional route, such as subcutaneous, oral, intramuscular, parenteral, or enteral route. In a preferred embodiment, the antibody is administered by an intraperitoneal route.

A candidate antibody can be administered to a non-human animal model in a single dose or in multiple doses. In a preferred embodiment, a single dose of antibody is administered to the non-human animal.

The candidate antibody can be administered to a non-human animal model during, prior to, or following testing in a fear conditioning assay. In one embodiment, a single dose of antibody is administered to a non-human animal model prior to testing in a fear conditioning assay. In a more preferred embodiment, a single dose of antibody is administered to a non-human animal model, approximately 24 hours prior to testing in a fear conditioning assay.

The candidate antibody can be administered at a dosage of about 0.1 mg to about 1000 mg of antibody per kg of body weight. More preferably, the antibody is administered at a dose of about 5 mg to about 50 mg of antibody per kg of body weight.

### V. COMBINATION STUDIES

Having identified a candidate immunotherapy based on its ability to reverse memory deficit and/or impairment in an animal model in a CFC assay one may combine this behavioral assessment with any one or more functional assessments of the immunotherapeutic, for example, any one or more in vitro and/or in vivo functional assessments. In vitro functions include but are not limited to binding of aggregated Aβ (e.g., aggregated Aβ1-42), binding to plaques, triggering phagocytosis (e.g., in an ex vivo phagocytosis assay), capturing soluble Aβ (e.g., soluble Aβ1-42). In vivo functions include but are not limited crossing the blood-brain barrier (BBB), reducing amyloid burden (e.g., clearing plaques), reducing neuritic dystrophy, maintaining synaptic architecture (e.g., maintaining synaptophysin). (See e.g., Bard et al. (2000) Nat. Med. 6:916-919; Bacskai et al. (2002) J. Neurosci. 22:7873-7878; Bard et al. (2003) PNAS USA 100:2023-2028). Moreover, one may combine this behavioral assessment with an analysis of the effects of treatment on the pathology of the animal model.

1. Testing Plaque Clearing Activity in Animal Models

Candidate immunotherapies may be assessed for plaque clearing activity. For example, model animals (e.g. PDAPP mice) may be injected with candidate anti-Aβ or specific anti-Aβ monoclonal, humanized, or chimeric antibodies. All antibody preparations are preferably purified to have low endotoxin levels. In an exemplary embodiment, mice are injected intraperitoneally as needed over a 4 month period to maintain a circulating antibody concentration measured by ELISA titer of greater than 1/1000 defined by ELISA to Aβ42 or other immunogen. Titers are monitored and mice are euthanized at the end of 6 months of injections. Histochemistry, Aβ levels and toxicology are performed post mortem. Ten mice are used per group.

2. Testing for Binding to Soluble Oligomeric A/3

Candidate passive immunotherapeutics, for example Aβ antibodies, may also be assessed directly for their ability to bind soluble oligomeric Aβ in a biochemical assay. The biochemical assay may be based, at least in part, on a comparison of the binding of the antibody to one or more forms of soluble, oligomeric Aβ (e.g., Aβ dimers, Aβ trimers, Aβ tetramers, Aβ pentamers, and the like) as compared to the binding of the antibody to monomeric Aβ. This comparison can be used to determine a relative binding of the antibody to soluble, oligomeric Aβ as compared to monomeric Aβ. In various embodiments, this relative binding is compared to a corresponding relative binding of a control reagent to one or more soluble oligomeric Aβ species versus monomeric Aβ. In other aspects, the affinity of an antibody for one or more oligomeric Aβ species is compared to the antibody's affinity for monomeric Aβ in the Aβ preparation. Candidate antibodies exhibit a preferential or greater binding to one or more soluble oligomeric Aβ species as compared to monomeric Aβ. Antibodies that preferentially bind to, for example, Aβ dimers, trimers and tetramers as compared to monomeric Aβ are preferred candidates for use in methods for effecting rapid improvement in cognition in a patient. For example, candidate antibodies exhibiting a two-fold, three-fold, four-fold, five-fold, ten-fold, twenty-fold or more greater binding to soluble oligomeric Aβ species as compared to monomeric Aβ are preferably selected for use in the therapeutic methods of the invention.

The binding of an antibody to one or more soluble, oligomeric Aβ species or to monomeric Aβ can be determined qualitatively, quantitatively, or combination of both. In general, any technique capable of distinguishing oligomeric Aβ species from monomeric Aβ in an Aβ preparation comprising the species can be used. In exemplary embodiments, one or more of immunoprecipitation, electrophoretic separation, and chromatographic separation (e.g., liquid chromatography), can be used to distinguish oligomeric Aβ species from monomeric Aβ in an Aβ preparation comprising the species.

In preferred embodiments, the binding of the antibody to one or more soluble, oligomeric Aβ species or to monomeric Aβ is determined by immunoprecipitating the Aβ species from the preparation. The immunoprecipitate is then subjected to an electrophoretic separation (e.g., SDS-PAGE) to distinguish oligomeric species from monomeric Aβ in the precipitate. The amount of oligomeric Aβ species and monomeric Aβ present in the electrophoretic bands can be visualized, for example, by immunoblotting of the electrophoretic gel or by direct quantitation of the respective species in the bands of the electrophoretic gel. The amount of precipitate for an Aβ species can be determined, for example, from the intensity of the corresponding electrophoretic bands, immunoblot bands, or a combination of both. The intensity determination can be qualitative, quantitative, or a combination of both.

Assessment of band intensity can be performed, for example, using appropriate film exposures which can be scanned and the density of bands determined with software, for example, AlphaEase™ software (AlphaInnotech™). Assessment of band intensity can be performed, for example, using any of a number of labels incorporated into the antibody, an imaging reagent (e.g., an antibody used in an immunoblot), or both. Suitable labels include, but are not limited to, fluorescent labels, radioactive labels, paramagnetic labels, or combinations thereof.

In various embodiments, the amount of one or more oligomeric Aβ species and/or monomeric Aβ which bind to an antibody can be assessed using mass spectrometry, for example, on the Aβ preparation itself a suitable time after it has been contacted with the antibody, or on monomeric Aβ and/or one or more soluble, oligomeric Aβ species bound to the antibody which have been extracted from the Aβ preparation.

In certain aspects, the affinity of an antibody for one or more oligomeric Aβ species is compared to the antibody's affinity for monomeric Aβ to identify the antibody as a candidate for use in the therapeutic methods of the invention, in particular, for use in method for effecting rapid improvement in cognition in a patient. The affinity of the test antibody (e.g., an Aβ antibody) for oligomeric Aβ as compared to monomeric AP can be compared to the binding affinities of a control reagent. Labels can be used to assess the affinity of an antibody for monomeric Aβ, oligomeric Aβ, or both. In various embodiments, a primary reagent with affinity for Aβ is unlabelled and a secondary labeling agent is used to bind to the primary reagent. Suitable labels include, but are not limited to, fluorescent labels, paramagnetic labels, radioactive labels, and combinations thereof.

In certain aspects, the methods of the invention feature the administration of an anti-Aβ antibody that is capable of rapidly improving cognition in a subject wherein the anti-Aβ antibody has been identified in using an assay which is suitably predictive of immunotherapeutic efficacy in the subject. In exemplary embodiments, the assay is a biochemical assay that is based, at least in part, on a comparison of the binding of one or more Aβ oligomers in an Aβ preparation to a test immunotherapeutic agent to the binding of Aβ monomers in the Aβ preparation to the test immunotherapeutic agent. The one or more Aβ oligomers can include, for example, one or more of Aβ dimers, Aβ trimers, Aβ tetramers, and Aβ pentamers. In various embodiments, the test immunotherapeutic agent is identified when the binding of one or more Aβ oligomers in the Aβ preparation to the test immunotherapeutic agent is greater than the binding of Aβ monomers in the Aβ preparation to the test immunotherapeutic agent. The amount of Aβ monomers and one or more Aβ oligomer species in an Aβ preparation which bind to a test immunological reagent can be assessed using biochemical methods, for example using immunoprecipitation to precipitate from the Aβ preparation the Aβ monomers and one or more Aβ oligomer species bound to the test immunological reagent followed by an electrophoretic separation of the immunoprecipitates. Such biochemical assays are discussed further herein and in copending U.S. Patent Application Ser. No. 60/636,687 (bearing Attorney Docket No. ELN-056-1, filed on Dec. 15, 2004), in copending U.S. Patent Application Ser. No. 60/736,045 (bearing Attorney Docket No. ELN-056-2, filed on Nov. 10, 2005), and in copending U.S. patent application Ser. No. ______ filed on even date herewith, the entire contents of which are hereby incorporated by reference.

3. Screening for Clearing Activity

Candidate immunotherapeutics may also be assessed directly in clearing an amyloid deposit or any other antigen, or associated biological entity, for which clearing activity is desired. To screen for activity against an amyloid deposit, a tissue sample from a brain of a patient with Alzheimer's disease or an animal model having characteristic Alzheimer's pathology is contacted with phagocytic cells bearing an Fc receptor, such as microglial cells, and the antibody under test in a medium in vitro. The phagocytic cells can be a primary culture or a cell line, and can be of murine (e.g., BV-2 or C8-B4 cells) or human origin (e.g., THP-1 cells). In some methods, the components are combined on a microscope slide to facilitate microscopic monitoring. In some methods, multiple reactions are performed in parallel in the wells of a microtiter dish. In such a format, a separate miniature microscope slide can be mounted in the separate wells, or a nonmicroscopic detection format, such as ELISA detection of Aβ can be used. Preferably, a series of measurements is made of the amount of amyloid deposit in the in vitro reaction mixture, starting from a baseline value before the reaction has proceeded, and one or more test values during the reaction. The antigen can be detected by staining, for example, with a fluorescently labeled antibody to Aβ or other component of amyloid plaques. A reduction relative to baseline during the reaction of the amyloid deposits indicates that the candidate immunotherapeutic has clearing activity. Such candidate immunotherapeutics are preferably employed in preventing or treating Alzheimer's and other amyloidogenic diseases.

### VI. PROPHYLACTIC AND THERAPEUTIC METHODS

Candidate immunotherapeutics discovered using the methods of the invention can be utilized for the treatment or prevention of cognitive disorders in a subject suffering from the disorder. Candidate immunotherapeutics are preferably administered to a patient under conditions that generate a beneficial therapeutic response in the patient (e.g., rapid improvement in cognition, induction of phagocytosis of Aβ, reduction of plaque burden, inhibition of plaque formation, reduction of neuritic dystrophy, and/or reversing, treating or preventing cognitive decline) in the patient, for example, for the prevention or treatment of the Aβ-related diseases or disorders or amyloidogenic diseases or disorders. Such diseases include Alzheimer's disease, Down's syndrome and mild cognitive impairment. The latter can occur with or without other characteristics of an amyloidogenic disease.

It will be appreciated by those in the art that the immunological reagents identified by the methods of the invention may be used to treat any disorder for which treatment with said immunological reagents is shown to provide a therapeutic benefit to a patient suffering from the disorder. For example, the disorder may be any cognitive disorder, e.g. a dementia disorder. Such cognitive deficits may have a number of origins: a functional mechanism (anxiety, depression), physiological aging (age-associated memory impairment), drugs, or anatomical lesions. Indications for which the immunotherapeutic agents discovered using methods of the inventions can be useful include learning disabilities or memory deficits due to toxicant exposure, brain injury leading to amnesia, age, schizophrenia, epilepsy, mental retardation, alcoholic blackouts, Korsakoff's syndrome, medication-induced amnesia (e.g. Halcion), basilar artery migraines, or amnesias associated with Herpes simplex encephalitis.

In an exemplary embodiment, immunotherapeutic agents discovered using the methods of the invention can be used to treat Alzheimer's and other amyloidogenic diseases such as Down's syndrome and cerebral amyloid angiopathy. The methods can be used on both asymptomatic patients and those currently showing symptoms of disease.

Therapeutic agents of identified by the methods of the invention are typically substantially pure from undesired contaminant. This means that an agent is typically at least about 50% w/w (weight/weight) pure, as well as being substantially free from interfering proteins and contaminants. Sometimes the agents are at least about 80% w/w and, more preferably at least 90 or about 95% w/w pure. However, using conventional protein purification techniques, homogeneous peptides of at least 99% w/w pure can be obtained.

In another aspect, an antibody identified by the methods of the invention may be administer to a paritient with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

a. Patients Amenable to Treatment

Patients amenable to treatment with the immunotherapies identified by the methods of the invention include individuals at risk of cognitive disorders but not showing symptoms, as well as patients presently showing symptoms. In the case of Alzheimer's disease, virtually anyone is at risk of suffering from Alzheimer's disease if he or she lives long enough. Therefore, immunotherapeutics identified by the present methods can be administered prophylactically to the general population without the need for any assessment of the risk of the subject patient.

The present methods are especially useful for individuals who are at risk for AD, e.g., those who exhibit risk factors of AD. The main risk factor for AD is increased age. As the population ages, the frequency of AD continues to increase. Current estimates indicate that up to 10% of the population over the age of 65 and up to 50% of the population over the age of 85 have AD.

Although rare, certain individuals can be identified at an early age as being genetically predisposed to developing AD. Individuals carrying the heritable form of AD, known as “familial AD” or “early-onset AD”, can be identified from a well documented family history of AD, of the analysis of a gene that is known to confer AD when mutated, for example the APP or presenilin gene. Well characterized APP mutations include the “Hardy” mutations at codons 716 and 717 of APP770 (e.g., valine⁷¹⁷ to isoleucine (Goate et al., (1991), Nature 349:704); valine⁷¹⁷ to glycine (Chartier et al. (1991) Nature 353:844; Murrell et al.(1991), Science 254:97); valine⁷¹⁷ to phenylalanine (Mullan et al.(1992), Nature Genet. 1:345-7)), the “Swedish” mutations at codon 670 and 671 of APP770, and the “Flemish” mutation at codon 692 of APP770. Such mutations are thought to cause Alzheimer's disease by increased or altered processing of APP to Aβ, particularly processing of APP to increased amounts of the long form of Aβ (i.e., Aβ1-42 and Aβ1-43). Mutations in other genes, such as the presenilin genes, PS1 and PS2, are thought indirectly to affect processing of APP to generate increased amounts of long form Aβ (see Hardy, TINS 20: 154 (1997); Kowalska et al., (2004), Polish J. Pharmacol., 56: 171-8). In addition to AD, mutations at amino acid 692 or 693 of the 770-amino acid isoform of APP has been implicated in cerebral amyloidogenic disorder called Hereditary Cerebral Hemorrhage with Amyloidosis of the Dutch-type (HCHW A-D).

More commonly, AD is not inherited by a patient but develops due to the complex interplay of a variety of genetic factors. These individuals are said to have “sporadic AD” (also known as “late-onset AD”), a form which is much more difficult to diagnose. Nonetheless, the patient population can be screened for the presence of susceptibility alleles or traits that do not cause AD but are known to segregate with AD at a higher frequency than in the general population, e.g., the ε2, ε3, and ε4 alleles of apolipoprotein E (Corder et. al. (1993), Science, 261: 921-923). In particular, patients lacking the ε4 allele, preferably in addition to some other marker for AD, may be identified as “at risk” for AD. For example, patients lacking the c4 allele who have relatives who have AD or who suffer from hypercholesterolemia or atherosclerosis may be identified as “at risk” for AD. Another potential biomarker is the combined assessment of cerebral spinal fluid (CSF) Aβ42 and tau levels. Low Aβ42 and high tau levels have a predictive value in identifying patients at risk for AD.

Other indicators of patients at risk for AD include in vivo dynamic neuropathological data, for example, in vivo detection of brain beta amyloid, patterns of brain activation, etc. Such data can be obtained using, for example, three-dimensional magnetic resonance imaging (MRI), positron emission tomography (PET) scan and single-photon emission computed tomography (SPECT). Indicators of patients having probable AD include, but are not limited to, patients (1) having dementia, (2) of an age of 40-90 years old, (3) cognitive deficits, e.g., in two or more cognitive domains, (4) progression of deficits for more than six months, (5) consciousness undisturbed, and/or (6) absence of other reasonable diagnoses.

Individuals suffering either sporadic or familial forms of AD are usually, however, diagnosed following presentation of one or more characteristic symptoms of AD. Common symptoms of AD include cognitive deficits that affect the performance of routine skills or tasks, problems with language, disorientation to time or place, poor or decreased judgment, impairments in abstract thought, loss of motor control, mood or behavior alteration, personality change, or loss of initiative. The number deficits or the degree of the cognitive deficit displayed by the patient usually reflects the extent to which the disease has progressed. For example, the patient may exhibit only a mild cognitive impairment, such that the patient exhibits problems with memory (e.g. contextual memory) but is otherwise able to function well.

Several tests have been developed to assess cognitive skills or performance in human subjects, for example, subjects at risk for or having symptoms or pathology of dementia disorders (e.g., AD). Cognitive deficits can be identified by impaired performance of these tests, and many treatments have been proposed based on their ability to improve performance in these tests. Although some tasks have evaluated behaviors or motor function of subjects, most tasks have been designed to test learning or memory.

Cognition in humans may be assessed using a wide variety of tests including, but not limited to, the following tests. The ADAS-Cog (Alzheimer Disease Assessment Scale-Cognitive) is 11-part test that takes 30 minutes to complete. The ADAS-Cog is a preferred brief exam for the study of language and memory skills. See Rosen et al. (1984) Am J Psychiatry. 141(11):1356-64; Ih1 et al. (2000) Neuropsychobiol. 41(2):102-7; and Weyer et al. (1997) Int Psychogeriatr. 9(2): 123-38.

The Blessed Test is another quick (˜10 minute) test of cognition which assesses activities of daily living and memory, concentration and orientation. See Blessed et al. (1968) Br J Psychiatry 114(512):797-811.

The Cambridge Neuropsychological Test Automated Battery (CANTAB) is used for the assessment of cognitive deficits in humans with neurodegenerative diseases or brain damage. It consists of thirteen interrelated computerized tests of memory, attention, and executive function, and is administered via a touch sensitive screen from a personal computer. The tests are language and largely culture free, and have shown to be highly sensitive in the early detection and routine screening of Alzheimer's disease. See Swainson et al. (2001) Dement Geriatr Cogn Disord. 12:265-280; and Fray and Robbins (1996) Neurotoxicol Teratol. 18(4):499-504. Robbins et al. (1994) Dementia 5(5):266-81.

The Consortium to Establish a Registry for Alzheimer's Disease (CERAD) Clinical and Neuropsychological Tests include a verbal fluency test, Boston Naming Test, Mini Mental State Exam (MMSE), ten-item word recall, constructional praxis, and delayed recall of praxis items. The test typically takes 20-30 minutes and is convenient and effective at assessing and tracking cognitive decline. See Morris et al. (1988) Psychopharmacol Bull. 24(4):641-52; Morris et al. (1989) Neurology 39(9):1159-65; and Welsh et al. (1991) Arch Neurol. 48(3):278-81.

The Mini Mental State Exam (MMSE) developed in 1975 by Folestein et al, is a brief test of mental status and cognition function. It does not measure other mental phenomena and is therefore not a substitute for a full mental status examination. It is useful in screening for dementia and its scoring system is helpful in following progress over time. The Mini-Mental State Examination MMSE is widely used, with norms adjusted for age and education. It can be used to screen for cognitive impairment, to estimate the severity of cognitive impairment at a given point in time, to follow the course of cognitive changes in an individual over time, and to document an individual's response to treatment. Cognitive assessment of subjects may require formal neuropsychological testing, with follow-up testing separated by nine months or more (in humans). See Folstein et al. (1975) J Psychiatr Res. 12:196-198; Cockrell and Folstein (1988) Psychopharm Bull. 24(4):689-692; and Crum et al. (1993) J. Am. Med. Association 18:2386-2391.

The Seven-Minute Screen is a screening tool to help identify patients who should be evaluated for Alzheimer's disease. The screening tool is highly sensitive to the early signs of AD, using a series of questions to assess different types of intellectual functionality. The test consists of 4 sets of questions that focus on orientation, memory, visuospatial skills and expressive language. It can distinguish between cognitive changes due to the normal aging process and cognitive deficits due to dementia. See Solomon and Pendlebury (1998) Fam Med. 30(4):265-71, Solomon et al. (1998) Arch Neurol. 55(3):349-55.

Individuals presently suffering from Alzheimer's disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of AD. Individuals suffering from Alzheimer's disease can also be diagnosed by ADRDA criteria as discussed in the Examples section.

b. Treatment Regimes and Dosages

In prophylactic applications, pharmaceutical compositions or medicaments are administered to a patient susceptible to, or otherwise at risk of, a dementia disorder in an amount sufficient to eliminate or reduce the associated learning and/or memory deficit, lessen the severity, or delay the outset of the disease, including biochemical, histologic and/or behavioral symptoms of the disease, its complications and intermediate pathological phenotypes presenting during development of the disease. In therapeutic applications, compositions or medicaments are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease.

In some methods, administration of an immunotherapeutic agent reduces or eliminates myocognitive impairment in patients that have not yet developed characteristic disease pathology. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. However, rapid improvement in cognition may be achieved following a single dose administration of antibody. The term “immune response” or “immunological response” includes the development of a humoral (antibody mediated) and/or a cellular (mediated by antigen-specific T cells or their secretion products) response directed against an antigen in a recipient subject. Such a response can be an active response, i.e., induced by administration of immunogen, or a passive response, i.e., induced by administration of immunoglobulin or antibody or primed T-cells. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages need to be titrated to optimize safety and efficacy.

The assay methods of the invention can be used to determine an effective dose range for use in humans by measuring both the LD50 (dose lethal to 50% of the non-human animal model) and the ED50 (the dose therapeutically effective in 50% of the population). The dose ratio between toxic and therapeutic effects is the therapeutic index and it can be expressed as the ratio LD50/ED50. Immunotherapeutic agents which exhibit large therapeutic indices are preferred. While agents that exhibit toxic side effects may be used, care should be taken to design a delivery system that targets the agent to the site of affected tissue in order to minimize potential damage to unaffected cells and, thereby, reduce side effects.

The data obtained from the non-human animal model assay can then be used to formulate a range of dosage for use in humans. The dosage of such agents lies preferably within a range of circulating concentration the include the ED50 with little or no toxicity. The dosage may vary within this range depending upon the dosage form employed and the route of administration utilized. A dose may be formulated to achieve a circulating plasma concentration range that includes the ED50 as determined for the non-human animal model. Such information can be used to more accurately determine useful doses in humans. Levels in plasma may be measured, for example, by high performance liquid chromatography.

For passive immunization with an antibody, the dosage ranges from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. In another example, dosages can be 0.5 mg/kg body weight or 15 mg/kg body weight or within the range of 0.5-15 mg/kg, preferably at least 1 mg/kg. In another example, dosages can be 0.5 mg/kg body weight or 20 mg/kg body weight or within the range of 0.5-20 mg/kg, preferably at least 1 mg/kg. In another example, dosages can be 0.5 mg/kg body weight or 30 mg/kg body weight or within the range of 0.5-30 mg/kg, preferably at least 1 mg/kg. In a preferred example, dosages can be about 30 kg/mg. In a particularly preferred example, the 12A11 antibody is administered intraperitoneally at a dose range from approximately 0.3 mg/kg to approximately 30 mg/kg.

Doses intermediate in the above ranges are also intended to be within the scope of the invention. Subjects can be administered such doses daily, on alternative days, weekly or according to any other schedule determined by empirical analysis. An exemplary treatment involves administration in multiple dosages over a prolonged period, for example, of at least six months. Additional exemplary treatment regimes involve administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Antibody is usually administered on multiple occasions. Intervals between single dosages can be weekly, monthly or yearly. Intervals can also be irregular as indicated by measuring blood levels of antibody to Aβ in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antibodies or a cocktail thereof are administered to a patient not already in the disease state. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the patient shows partial or complete amelioration of symptoms of disease. Thereafter, the patient can be administered a prophylactic regime.

Doses for nucleic acids encoding antibodies range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. Preferred methods for administering an immunogenic agent include intramuscular or subcutaneous injection. Intramuscular injection is most typically performed in the arm or leg muscles. In some methods, agents are injected directly into a particular tissue where deposits have accumulated, for example intracranial injection. Intramuscular injection or intravenous infusion are preferred for administration of antibody. In some methods, antibodies are administered as a sustained release composition or device, such as a Medipad™ device.

Agents identified by the methods of the invention can optionally be administered in combination with other agents that are at least partly effective in treatment of amyloidogenic disease. In certain embodiments, a humanized antibody (e.g., humanized 12A11) is administered in combination with a second immunogenic or immunologic agent. For example, a candidate Aβ antibody can be administered in combination with another humanized antibody to Aβ. In other embodiments, a candidate Aβ antibody is administered to a patient who has received or is receiving an Aβ immunogen. In the case of Alzheimer's and Down's syndrome, in which amyloid deposits occur in the brain, agents identified by the methods of the invention can also be administered in conjunction with other agents that increase passage of said agents across the blood-brain barrier. Agents identified by the methods of the invention can also be administered in combination with other agents that enhance access of the therapeutic agent to a target cell or tissue, for example, liposomes and the like. Co-administering such agents can decrease the dosage of a therapeutic agent (e.g., therapeutic antibody or antibody chain) needed to achieve a desired effect.

c. Pharmaceutical Compositions

Agents identified by the methods of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose (TM), agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, agents identified by the methods of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents identified by the methods of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25:3521 (1995); Cevc et al., Biochem. Biophys. Acta 1368:201-15 (1998)).

The present invention will be more fully described by the following non-limiting examples.

### EXAMPLES

Methods & Materials

Testing Apparatus

Transgenic mice and wild-type littermate control mice were individually housed for at least 2 weeks prior to any testing and allowed ad libitum access to food and water. CFC occurred in six 30×24×21 cm operant chambers (Med Associates, Inc) constructed from aluminum sidewalls and plexiglass ceiling, door and rear wall. Each chamber was equipped with a floor consisting of 36 stainless steel rods through which a foot shock could be administered. In addition, each chamber had 2 stimulus lights, one house light and a solenoid. Lighting, the footshock (US) and the solenoid (CS) were all controlled by a PC running MED-PC software. The chambers were located in a sound isolated room in the presence of red light.

CFC Assay

Mice (n=8-12/genotype/treatment) were trained and tested on two consecutive days. The Training Phase consisted of placing the mice in the operant chambers, illuminating both the stimulus and houselights and allowing them to explore for 2 minutes. At the end of the two minutes, a footshock (US; 1.5 mAmp) was administered for 2 seconds. This procedure was repeated and 30 seconds after the second foot shock the mice were removed from the chambers and returned to their home cages.

Twenty hours after training, animals were returned to the chambers in which they had previously been trained. Freezing behavior, in the same environment in which they had received the shock (“Context”), was then recorded by the experimenter using time sampling in 10 seconds bins for 5 minutes (30 sample points). Freezing was defined as the lack of movement except that required for respiration. At the end of the 5 minute Context test mice were returned to their home cages.

### Example I

### Effect of Passive Immunization with Aβ40 mAbs on Contextual Memory of the Tg2576 Mouse

Approximately 20-week old wild-type mice and Tg2576 transgenic mice were administered a single dose of phosphate buffered saline (PBS) or treatment antibody by intraperitoneal injection at 24 hours prior to the training phase of the CFC. Treatment antibodies were raised to N-terminal, central, and C-terminal portions of the Aβ peptide.

Transgenic (Tg2576) mice were heterozygous for the K670N/M671L transgene. All transgenic genotypes were confirmed by PCR and all animals homozygous for the Retinal Degeneration (Rd) mutation were excluded. The background strain consisted of a C57B16 and 129SJL cross. Tg2576 mice exhibited cognitive deficits in contextual memory beginning at 14-16 weeks of age. Cognitive deficits were particularly prominent at 20 weeks of age and were maintained up to 65 weeks of age.

Results:

i) N-Terminal Antibodies

The therapeutic efficacy of several mAbs raised against the N-terminus of Aβ (e.g. Aβ residues 1-7) are tabulated in Table 1. Therapeutic efficacy is expressed both in terms of memory deficit reversal and memory impairment status. “Memory deficit reversal” was determined by comparing the freezing behavior of mAb-vs. PBS control-treated Tg2576 animals. “Memory impairment status” was determined by comparing the freezing behavior of Wild-type vs. Tg2576 mAb-treated animals. The results indicated that the mAbs 3D6, 10D5, and 12A11 caused a significant (**) improvement in contextual memory of Tg2576 mice relative to a control treatment (p value<0.05) (ie. significant memory deficit reversal), and no significant memory impairment (##) with respect to wild-type mice (p value>0.1). A further N-terminal mAb, 3A3, was also found to be efficacious at reversing cognitive memory deficits in Tg2576 mice in the CFC assay. Furthermore, the antibodies 6C6, 10D5, and 12B4 displayed a trend towards either memory deficit reversal (*) or no memory Impairment (#) (0.1>p value>0.05).

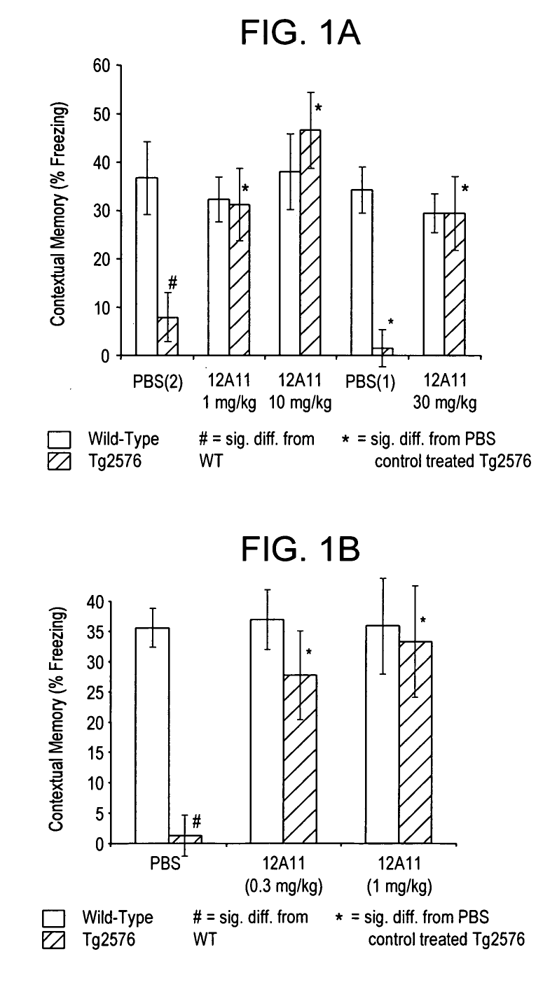

Tg2576 mice displayed prominent and significant memory deficit reversal when administered the N-terminal, murine IgG2a mAb designated 12A11, and this mAb was effective treatment at every dose tested (0.3, 1, 10, or 30 mg/kg) (see FIGS. 1A and 1B). Untreated (PBS) Tg2576 mice displayed a significant deficit in contextual-dependent memory (#) (ie. a significant memory impairment status) in comparison with wild-type mice (FIG. 1A). However, Tg2576 mice exhibited a full and significant memory deficit reversal (*) when administered 1, 10, or 30 mg/kg (i.p.) of 12A11. The improvement in cognitive performance persisted when mice were administered lower doses (0.1 and 1 mg/kg i.p.) of 12A11 (FIG. 1B). To confirm that the observed response was due to amyloid binding, Tg2576 mice were administered 30 mg/kg of IgG2a isotype control mAb raised against an unrelated antigen from E. tennela. As expected, Tg2576 mice treated with the control antibody exhibited profound defects in contextual memory in relation to wild-type mice (data not shown).

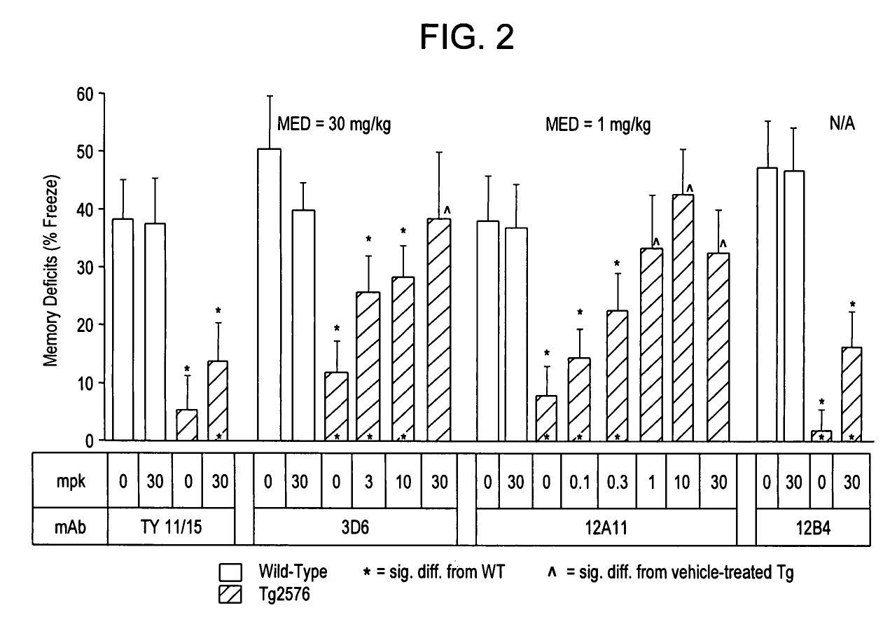

In another experiment, the effect of the N-terminal antibodies 3D6, 12A11, and 12B4 were compared directly in a CFC assay with Tg2576 mice (see FIG. 2). Consistent with previous results, 12A11 induced a prominent and significant memory deficit reversal at 1, 10, or 30 mg/kg (ˆ). Morever, 3D6 induced significant memory deficit reversal at 30 mg/kg. In contrast, both 12B4 antibodies and an unrelated IgG1 antibody (TY 11/15) failed to induce significant memory deficit reversal.

TABLE 1
Effect of N-terminal Aβ mAbs on Contextual Memory of Tg2576 mice

| | Memory Deficit Reversal | | | | | Impairment Status | | | | |
|---|---|---|---|---|---|---|---|---|---|---|
| | per Ab dosage (mg/kg) | | | | | per Ab dosage (mg/kg) | | | | |
| mAb | (p value WRT PBS Control) | | | | | (p value WRT WT mice) | | | | |
| tested | 0.3 | 1 | 3 | 10 | 30 | 0.3 | 1 | 3 | 10 | 30 |
| 3D6 | | | 0.3680 | 0.1586 | 0.0004## | | | 0.0529* | 0.2585** | 0.8972** |
| 6C6 | | | | | 0.0588# | | | | | 0.0056 |
| 6C6^{†} | | | | | 0.6567 | | | | | 0.0088 |
| 10D5 | | | 0.7045 | 0.9661 | 0.0189## | | | 0.0009 | 0.002 | 0.0752* |
| 2H3 | | | | | 0.3007 | | | | | 0.1333** |
| 12B4 | | | | | 0.1122 | | | | | 0.0013 |
| 12B4^{†} | | | | | 0.1015 | | | | | 0.756* |
| 12A11 | | 0.02## | | 0.0002## | 0.0007## | | 0.9092** | | 0.3838** | 0.9901** |
| 12A11^{†} | 0.0055## | 0.001## | | | | 0.3341** | 0.7773** | | | |

^{†}Indicates a repeat experiment performed with a different preparation of the same antibody

ii) Central Antibodies

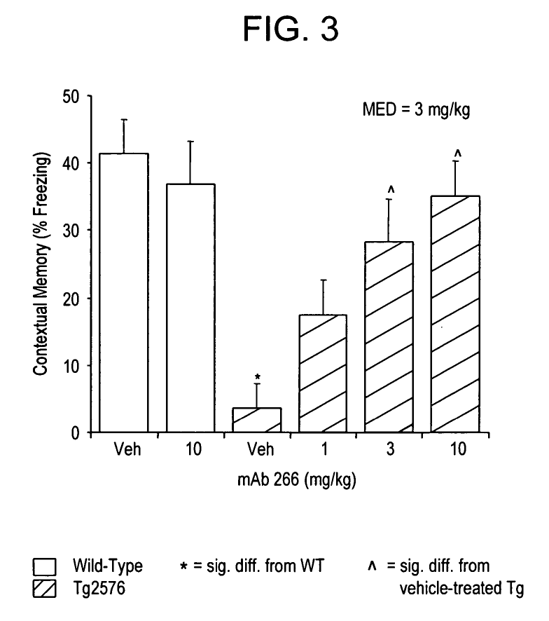

The therapeutic efficacy of several mAbs raised against the central amino acid sequence (e.g. Aβ amino acid residues 16-24) of Aβ are tabulated in Table 2. The results indicate that the mAbs 266 and 15C11 caused a significant improvement in contextual memory (**)(ie. significant memory deficit reversal) of Tg2576 mice relative to control treatment (p value<0.05), and no significant memory impairment (##) with respect to wild-type mice (p value>0.1). Furthermore, the antibodies 1C2 and 2B1 displayed a trend towards memory deficit reversal (*). (0.1>p value>0.05). The efficacy of, for example, mAb266, is also depicted graphically in FIG. 3.

TABLE 2
Effect of Central anti-Aβ mAbs on Contextual Memory in Tg2576 mice

| | Memory Deficit Reversal | | | | Impairment Status | | | |
|---|---|---|---|---|---|---|---|---|
| | (p value WRT PBS control) | | | | (p value WRT WT mice) | | | |
| | 1 | 3 | 10 | 30 | 1 | 3 | 10 | 30 |
| mAb | (mg/kg) | (mg/kg) | (mg/kg) | (mg/kg) | (mg/kg) | (mg/kg) | (mg/kg) | (mg/kg) |
| 266 | 0.1269 | 0.0082** | 0.0002** | | 0.1635## | 0.1084## | 0.8348## | |
| 6H9 | | | | 0.1122 | | | | 0.0044 |
| 1C2 | | | | 0.0695* | | | | 0.0626# |
| 15C11 | | 0.1246 | 0.1156 | 0.0274** | | 0.6228## | 0.3399 ## | 0.8907## |
| 2B1 | | | | 0.0578* | | | | 0.4020## |

Cell lines producing the antibodies 1C2, 2B1, 6C6 and 9G8, having the the A TCC accession numbers ______, ______, and ______, respectively, were deposited on Oct. 31, 2005 under the terms of the Budapest Treaty.

iii) C-Terminal Antibodies

The therapeutic efficacy of several mAbs raised against the carboxy terminal amino acid sequence of Aβ is tabulated in Table 3. The results indicate that the most antibodies raised against the C-terminus of Aβ were relatively ineffective in treating cognitive impairment at the single dose tested (30 mg/kg). Of the four monoclonals tested, none produced any improvement in the contextual memory of Tg2576 mice relative to control treatment (p value>0.1), although three (2G3, 14C2, and 16C 1) displayed a trend toward no impairment (#) with respect to wild-type mice (0.1>p value>0.05).

TABLE 3
Effect of C-terminal anti-Aβ mAbs
on Contextual Memory in Tg2576 mice

| | Memory Deficit Reversal | | | | Impairment Status | | | |
|---|---|---|---|---|---|---|---|---|
| | (p value WRT PBS control) | | | | (p value WRT WT mice) | | | |
| | 1 | 3 | 10 | | 1 | 3 | 10 | |
| | (mg/ | (mg/ | (mg/ | 30 | (mg/ | (mg/ | (mg/ | 30 |
| mAb | kg) | kg) | kg) | (mg/kg) | kg) | kg) | kg) | (mg/kg) |
| 2G3 | | | | 0.7521 | | | | 0.0589# |
| 14C2 | | | | 0.79654 | | | | 0.0719# |
| 21F12 | | | | 0.2026 | | | | 0.0151 |
| 16C11 | | | | 0.1523 | | | | 0.0985# |

### Example II

### Effect of Passive Immunization with AP40 mAbs on Contextual Memory of 18-20 Month Old, Plaque-bearing AD Mice

Wild-type mice and transgenic AD mice were administered a single dose of phosphate buffered saline (PBS) or treatment antibody (C-terminal 266 antibody or N-terminal 12A11 antibody) by intraperitoneal injection at 24 hours prior to the training phase of the CFC. The transgenic AD mice used in the experiment were approximately 18-20 months of age and displayed prominent cognitive defects, as well a dense accumulation of plaque.

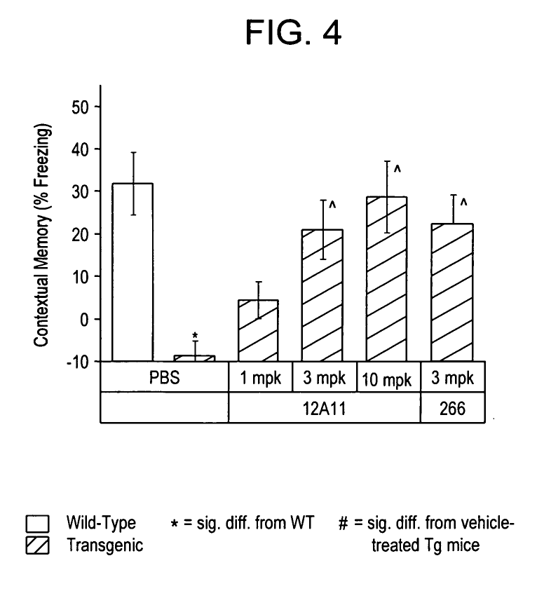

Transgenic mice displayed prominent and significant reversal of contextual memory deficit when administered the N-terminal, murine IgG2a mAb designated 12A11, and this mAb was effective treatment at several low doses (3 and 10 mg/kg). (see FIG. 4). Low-dose treatment with the central terminal antibody designated 266 also resulted in a significant reversal of the contextual-memory deficit in transgenic mice. In contrast, untreated (PBS) transgenic mice displayed a significant deficit in contextual-dependent memory (ie. a significant memory impairment status) in comparison with wild-type mice (*). These results demonstrate that acute reversal of contextual memory deficits is maintained in aged mice exhibiting prominent Alzheimer's disease pathology (AD).

Although the foregoing invention has been described in detail for purposes of clarity of understanding, it will be obvious that certain modifications may be practiced within the scope of the appended claims. All publications and patent documents cited herein, as well as text appearing in the figures and sequence listing, are hereby incorporated by reference in their entirety for all purposes to the same extent as if each were so individually denoted.

What is claimed is:

1. A method for identifying an immunotherapeutic agent effective for improving cognition in a subject suffering from a cognitive disorder, comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
whereby an improvement in context-dependent memory identifies the immunotherapeutic agent as effective for improving cognition in the subject.

2. A method for identifying an immunotherapeutic agent effective for improving cognition in a subject suffering from a cognitive disorder, comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to an appropriate control,
whereby an improvement in context-dependent fear response identifies the immunotherapeutic agent as effective for improving cognition in the subject.

3. The method of claim 1 or 2, wherein the cognitive disorder is an amyloidogenic disorder.

4. The method of claim 3, wherein the amyloidogenic disorder is Alzheimer's disease.

5. The method of claim 1 or 2, wherein the model animal is selected from the group consisting of a PDAPP mouse, a Tg2576 mouse, a TgAPP22 mouse, a TgAPP/LD/2 mouse, a PSEN-1 A246E mouse, a PSEN-1 DeltaE9 mouse, a Tg2576+PSEN-1 mouse, a TgHu/MoAPP A246E +PSEN-1 mouse, a TgHu/MoAPP DeltaE9+PSEN-1 mouse, a TgCDNR8 mouse, a PSAPP mouse, and a 3xTg-AD mouse.

6. The method of claim 1 or 2, wherein the model animal is at least 20 weeks of age prior to administration of the test immunotherapeutic agent.

7. The method of claim 1 or 2, wherein the model animal is at least 10 weeks of age prior to administration of the test immunotherapeutic agent.

8. The method of claim 1 or 2, wherein the cognitive deficit is an impairment in procedural learning and/or memory.

9. The method of claim 8, wherein the impairment in procedural learning and/or memory is contextual-dependent.

10. The method of claim 8, wherein the impairment in procedural learning and/or memory is cue-dependent.

11. The method of claim 1 or 2, wherein the aversive stimulus is a footshock.

12. The method of claim 1 or 2, wherein two or fewer training sessions are suitable to condition the model animal to the aversive stimulus.

13. The method of claim 1, wherein the model animal is administered a cue-dependent stimulus paired with the aversive stimulus during the training session.

14. The method of claim 13, wherein the model animal is administered a cue-dependent stimulus in the absence of the aversive stimulus during the testing session.

15. The method of claim 13 or 14, wherein the cue conditioning stimulus is an auditory cue.

16. The method of claim 1 or 2, wherein the context conditioning stimulus is an altered cage.

17. The method of claim 2, wherein the fear response is a freezing behavior.

18. The method of claim 2, wherein the suitable control is a wild-type animal administered the test immunotherapeutic.

19. The method of claim 18, wherein the improvement in the context-dependent fear response is a nonsignificant difference in status of impairment of the model animal as compared to the wild-type animal.

20. The method of claim 2, wherein the suitable control is a model animal that is not administered the test immunotherapeutic agent.

21. The method of claim 20, wherein the improvement in the context-dependent fear response is a significant difference in deficit reversal as compared to the model animal that is not administered the test immunotherapeutic agent.

22. The method of claim 1 or 2, wherein steps (i), (ii), (iii), or (iv) are repeated one to five times.

23. The method of claim 1 or 2, wherein the model animal is administered multiple doses of a test immunotherapeutic agent.

24. The method of claim 22, wherein steps (i), (ii), (iii), or (iv) are repeated with a different concentration of the test immunotherapeutic agent.

25. The method of claim 1 or 2, wherein step (iii) is performed within 24 hours of step (i).

26. The method of claim 1 or 2, wherein the test immunotherapeutic agent is a passive immunotherapeutic agent.

27. The method of claim 26, wherein the test immunotherapeutic agent is an Aβ antibody.

28. The method of claim 27, wherein the Aβ antibody is administered in a single dose.

29. The method of claim 1 or 2, wherein the test immunotherapeutic agent is selected from the group consisting of a humanized Aβ antibody, a chimeric Aβ antibody, and a variant Aβ antibody, or antigen binding fragments thereof.

30. The method of claim 29, wherein the test immunotherapeutic agent is administered in a single dose.

31. A method for identifying an immunotherapeutic agent effective in neutralizing one or more toxic soluble forms of Aβ peptide, comprising the steps of:
(i) administering a test immunotherapeutic agent to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
whereby an improvement in context-dependent memory identifies the immunotherapeutic agent as effective in neutralizing one or more toxic soluble forms of Aβ peptide.

32. The method of claim 31, wherein the test immunotherapeutic agent is further tested for its ability to bind to and/or clear insoluble forms of Aβ peptide.

33. The method of claim 32, whereby a greater than 50% reduction in the size and number of amyloid deposits identifies the immunotherapeutic agent as effective in clearing plaque.

34. A method of identifying an epitope in an Aβ peptide comprising the steps of:
(i) administering a fragment of the Aβ peptide to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus in the absence of the aversive stimulus,
whereby an improvement in context-dependent memory identifies an epitope in the Aβ peptide.

35. The method of claim 34, wherein the method is repeated with a truncated form of the Aβ peptide.

36. The method of claim 35, wherein the Aβ peptide is used as an active immunotherapeutic agent for improving cognition in a subject.

37. The method of claim 35, wherein the Aβ peptide used to generate an antibody.

38. The method of claim 37, wherein the antibody is used as a passive immunotherapeutic agent for improving cognition in a subject.

39. A method for identifying an immunotherapeutic agent effective for improving cognition in a subject suffering from a cognitive disorder, the method comprising the steps of:
(i) administering an Aβ peptide to a model animal of an amyloidogenic disorder wherein the model animal exhibits a cognitive deficit;
(ii) conducting at least one training session in which the model animal is administered a context-dependent stimulus that is paired with an aversive stimulus; and
(iii) conducting at least one testing session in which the model animal is administered a context-dependent stimulus when the in vivo concentration of the test immunotherapeutic agent is more than 50% of the dose administered in step (i),
whereby an improvement in context-dependent memory identifies the immunotherapeutic agent as effective in improving cognition in the subject.

40. A method for identifying an immunotherapeutic agent effective for improving cognition in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a wild-type animal administered the test immunotherapeutic,
whereby a nonsignificant difference in status of impairment of the model animal as compared to the wild-type animal identifies the test immunotherapeutic agent as effective in improving cognition in the subject.

41. A method for identifying an immunotherapeutic agent effective for improving cognition in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a model animal that is not administered the test immunotherapeutic agent,
whereby a significant difference in deficit reversal of the model animal in step (iii) as compared to the model animal that is not administered the test immunotherapeutic agent identifies the test immunotherapeutic agent as effective in improving cognition.

42. A method for identifying an immunotherapeutic agent effective for improving cognition in a subject suffering from a cognitive disorder, the method comprising the steps of:
(i) administering a test immunotherapeutic agent to a model animal of the disorder wherein the model animal exhibits a cognitive deficit;
(ii) conducting at least one training session in which the model animal is administered a context-dependent stimulus that is paired with an aversive stimulus;
(iii) conducting at least one testing session in which the model animal is administered a context-dependent stimulus in the absence of the aversive stimulus; and
(iv) comparing a context-dependent fear response of the model animal in step (iii) to a context-dependent fear response of a wild-type animal administered the test immunotherapeutic;
(v) comparing a context-dependent fear response of the model animal in step (iii) to a context-dependent fear response of a model animal that is not administered the test immunotherapeutic agent,
whereby a nonsignificant difference in status of impairment of the model animal as compared to the wild-type animal and a significant difference in deficit reversal of the model animal in step (iii) as compared to the model animal that is not administered the test immunotherapeutic agent identifies the test immunotherapeutic agent as effective in improving cognition.
